(19) United States
(12) Patent Application Publication
(10) Pub. No.: US 2026/0090189 A1
(43) Pub. Date: Mar. 26, 2026

(54) ORGANIC COMPOUND AND SYNTHESIS METHOD THEREOF, AND OPTOELECTRONIC DEVICE

(71) Applicants: Guangdong Juhua Research Institute of Advanced Display, Guangzhou (CN); TCL Technology Group Corporation, Huizhou (CN)

(72) Inventor: Mingshu LIU, Guangzhou (CN)

(73) Assignees: Guangdong Juhua Research Institute of Advanced Display, Guangzhou (CN); TCL Technology Group Corporation, Huizhou (CN)

(21) Appl. No.: 19/215,070

(22) Filed: May 21, 2025

(30) Foreign Application Priority Data
May 24, 2024 (CN) 202410663166.5

(51) Int. Cl. H10K 50/115; C07C 233/42; H10K 50/15; H10K 50/16; H10K 50/17; H10K 85/60

(52) U.S. Cl. CPC H10K 50/115; C07C 233/42; H10K 50/15; H10K 50/16; H10K 50/17; H10K 85/631

(57) ABSTRACT

An organic compound and synthesis method thereof, and an optoelectronic device. The organic compound includes a compound represented by the general formula (1):

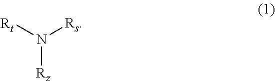

By disposing a film including the organic compound between a hole functional layer and an electron functional layer of an optoelectronic device, the luminous efficiency of the optoelectronic device may be improved, and the lifetime of the optoelectronic device may be prolonged.

This application claims priority to Chinese Application No. 202410663166.5, entitled “ORGANIC COMPOUND A AND SYNTHESIS METHOD THEREOF, OPTOELECTRONIC DEVICE, AND DISPLAY DEVICE”, filed on May 24, 2024. The entire disclosures of the above application are incorporated herein by reference.

### TECHNICAL FIELD

The present disclosure relates to a field of optoelectronic device technologies, and more particularly, to an organic compound and synthesis method thereof, and an optoelectronic device.

### BACKGROUND

Nowadays, the widely used QLED has the advantages of saturated color of emitted light, adjustable wavelength, low lighting voltage, good solution processability, and easy fine control of quantum dots.

Conventional QLED device generally include an anode, a hole injection layer, a hole transport layer, a light-emitting layer, an electron transport layer, an electron injection layer, and a cathode. Under the action of the electric field, the holes generated by the anode and the electrons generated by the cathode of the optoelectronic device move and inject into the hole transport layer and the electron transport layer respectively, and finally migrate to the light-emitting layer. When the two meet in the light-emitting layer, energy excitons are generated, thereby exciting the light-emitting molecules to finally produce visible light.

The luminous efficiency of the prior photoelectric device is low and needs to be further improved.

### Technical Solution

The present disclosure provides an organic compound and synthesis method thereof, and an optoelectronic device.

First aspect, embodiments of the present disclosure provide an organic compound including a compound represented by the general formula (1):

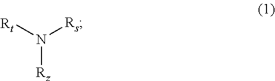

where, each time R_{z}, Rₛ and Rₜ appear are independently selected from

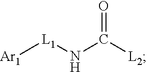

each time Ar₁ appears is independently selected from an aryl group having 6 to 30 ring atoms that is unsubstituted or substituted with at least one R, or a heteroaryl group having 5 to 30 ring atoms that is unsubstituted or substituted with at least one R, or any combination of these groups; a heteroatom of the heteroaryl group is selected from one or more of N, S, O, P, and Si, and a number of the heteroatom of the heteroaryl group is selected from 1 to 20;
each time L₁ appears is independently selected from a single bond, a C₁-C₃₀ aliphatic chain hydrocarbon group unsubstituted or substituted with at least one R, a hydroxyl group, an aldehyde group, a sulfhydryl group, an ether group, an ester group, an amino group, a carbonyl group, a nitro group, or a halogen;
each time L₂ appears is independently selected from a C₁-C₃₀ aliphatic chain hydrocarbon group unsubstituted or substituted with at least one R, a hydroxyl group, an aldehyde group, a sulfhydryl group, an ether group, an ester group, an amino group, a carbonyl group, or a nitro group; and
each time R appears is independently selected from deuterium, a halogen, a hydroxyl group, a carboxyl group, a nitro group, a sulfonyl group, a sulfhydryl group, a cyano group, an aldehyde group, an amide group, or any combination of these groups.

Second aspect, embodiments of the present disclosure further provide a method of synthesizing an organic compound, the method includes:
mixing a first compound and a second compound in a first solvent to react to obtain an organic compound;
a structure of the first compound is represented by the general formula (I):

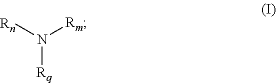

where each time Rₘ, Rₙ and R_{q} appear are each independently selected from

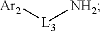

each time Ar₂ appears is independently selected from an aryl group having 6 to 30 ring atoms that is unsubstituted or substituted with at least one R, or a heteroaryl group having 5 to 30 ring atoms that is unsubstituted or substituted with at least one R, or any combination of these groups; a heteroatom of the heteroaryl group is selected from one or more of N, S, O, P, and Si, and a number of the heteroatom of the heteroaryl group is selected from 1 to 20;
each time L₃ appears is independently selected from a single bond, a C₁-C₃₀ aliphatic chain hydrocarbon group unsubstituted or substituted with at least one R, a hydroxyl group, an aldehyde group, a sulfhydryl group, an ether group, an ester group, an amino group, a carbonyl group, a nitro group, or a halogen;
each time R appears is independently selected from deuterium, a halogen, a hydroxyl group, a carboxyl group, a nitro group, a sulfonyl group, a sulfhydryl group, a cyano group, an aldehyde group, an amide group, or any combination of these groups;
a structure of the second compound is represented by the general formula (II):

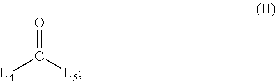

where L₄ is selected from a C₁-C₃₀ aliphatic chain hydrocarbon group unsubstituted or substituted with at least one R, a hydroxyl group, an aldehyde group, a sulfhydryl group, an ether group, an ester group, an amino group, a carbonyl group, or a nitro group;
L₅ is selected from a halogen; and
each time R appears is independently selected from deuterium, a halogen, a hydroxyl group, a carboxyl group, a nitro group, a sulfonyl group, a sulfhydryl group, a cyano group, an aldehyde group, an amide group, or any combination of these groups.

Third aspect, embodiments of the present disclosure further provide an optoelectronic device including a first electrode, a functional layer, and a second electrode stacked in this order; wherein the functional layer includes the organic compound described above.

In the embodiments of the present disclosure, by disposing a film including the organic compound in the optoelectronic device, it is possible to realize improvement of the surface of adjacent films, increasing the luminous efficiency of the optoelectronic device, and prolonging the lifetime of the optoelectronic device.

### BRIEF DESCRIPTION OF DRAWINGS

In order to more clearly explain the technical solutions in the embodiments of the present disclosure, the figures to be used in the description of the embodiments are briefly described below. It is apparent that the figures in the following description are merely some embodiments of the present disclosure. For those skilled in the art, without involving any creative effort, other figures may be obtained based on these figures.

FIG. 1 is a structure schematic diagram of an optoelectronic device according to an embodiment of the present disclosure;

FIG. 2 is a flowchart of a method of synthesising an organic compound according to an embodiment of the present disclosure;

FIG. 3 is a flowchart of another method of synthesising an organic compound according to an embodiment of the present disclosure.

### DETAILED DESCRIPTION

Technical solutions in embodiments of the present disclosure will be clearly and completely described below with reference to the drawings in the embodiments of the present disclosure. It is apparent that, the described embodiments are only a part of embodiments of the present disclosure, rather than all the embodiments. Based on the embodiments in the present disclosure, all other embodiments obtained by those skilled in the art without creative effort fall within the protection scope of the present disclosure.

Furthermore, it should be understood that the detailed description described herein is for illustration and explanation of the present disclosure only, and is not intended to limit the present disclosure.

In the present disclosure, unless stated to the contrary, the location words used such as “upper” and “lower” usually refer to the upper and lower in the actual use or working state of the device, specifically the drawing direction in the accompanying figures; while “inner” and “outer” are for the outline of the device. In addition, in the description of the present disclosure, the term “comprising/including “means” comprising/including but not limited to”. The terms first, second, third, etc. are used for indication only, and do not impose numerical requirements or establish order.

In the present disclosure, the term “and/or” is used to describe the association of associated objects, and means that there may be three relationships, for example, “A and/or B” may refer to three cases: the first case refers to the presence of A alone; the second case refers to the presence of both A and B; the third case refers to the presence of B alone, where A and B may be singular or plural.

In the present disclosure, the term “at least one” refers to one or more, and “a plurality of/multiple” refers to two or more. The terms “at least one”, “at least one of the followings”, and the like, refer to any combination of the items listed, including any combination of the singular or the plural items. For example, “at least one of a, b, or c” or “at least one of a, b, and c” may refer to: a, b, c, a-b (i.e., a and b), a-c, b-c, or a-b-c, where a, b, and c may be single or plural (multiple).

In the present disclosure, another layer is formed “on” a certain layer, and the so-called “on” is a broad concept, which may mean that the other layer is formed adjacent to the certain layer, or may mean that another spacer structure layer exists between the other layer and the certain layer. For example, a second electrode is formed “on” the first carrier functional layer, and the so-called “on” may mean that the second electrode is formed adjacent to the first carrier functional layer, or may mean that another spacer structure layer exists between the second electrode and the first carrier functional layer, for example, a light-emitting layer.

Various embodiments of the present disclosure may be presented in a form of range. It should be understood that the description in the form of range is merely for convenience and brevity, and should not be construed as a hard limitation on the scope of the disclosure. Therefore, it should be considered that the recited range description has specifically disclosed all possible subranges, as well as a single numerical value within that range. For example, it should be considered that a description of a range from 1 to 6, more specifically, a range such as from 1 to 3, from 1 to 4, from 1 to 5, from 2 to 4, from 2 to 6, from 3 to 6, etc., and a single number within the range, such as 1, 2, 3, 4, 5, and 6, regardless of the range.

The present disclosure provides an organic compound, a method of synthesizing the organic compound, and an optoelectronic device. In order to make the object, technical solution, and effect of the present disclosure more clearly, the present disclosure will be described in further detail below. It should be understood that the specific embodiments described herein are merely for illustration of the present disclosure, and are not intended to limit the present disclosure. The technical solution of the present disclosure is as follows:

In the present disclosure, “substituted” means that a hydrogen atom in a substituted group is substituted with a substituent.

In the present disclosure, when the same substituent occurs multiple times, it may be independently selected from different groups. If the general formula contains a plurality of R¹, R¹ may be independently selected from different groups. For example, the three R¹ in the general formula

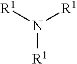

may be the same as or different from each other.

In the present disclosure, “substituted or unsubstituted” means that the defined group may or may not be substituted. When the defined group is substituted, it should be understood that the defined group may be substituted with one or more substituents R, and the R may be selected from, but not limited to, deuterium atom, a cyano group, an isocyano group, nitro group, halogen, an alkyl having 1 to 30 carbon atoms, a heterocyclyl having 3 to 20 ring atoms, an aromatic group having 6 to 20 ring atoms, a heteroaromatic group having 5 to 20 ring atoms, —NR′R″, silyl, carbonyl, alkoxycarbonyl, aryloxycarbonyl, carbamoyl, haloformyl, formyl, isocyanate, thiocyanate, isothiocyanate, hydroxyl group, trifluoromethyl, and the above groups may be further substituted with art acceptable substituents. It should be understood that R′ and R″ in —NR′R″ are each independently selected from, but not limited to, hydrogen atom, deuterium atom, a cyano group, an isocyano group, a nitro group, a halogen, an alkyl group having 1 to 10 carbon atoms, a heterocyclyl group having 3 to 20 ring atoms, an aromatic group having 6 to 20 ring atoms, and a heteroaromatic group having 5 to 20 ring atoms.

In at least one embodiment, R′ and R″ are each independently selected from, but not limited to, deuterium atom, a cyano group, an isocyano group, nitro group, halogen, an alkyl group having 1 to 10 C atoms, a heterocyclic group having 3 to 10 ring atoms, an aromatic group having 6 to 20 ring atoms, a heteroaromatic group having 5 to 20 ring atoms, silyl group, carbonyl group, alkoxycarbonyl group, aryloxycarbonyl group, carbamoyl group, haloformyl group, formyl group, isocyanate group, thiocyanate group, isothiocyanate group, hydroxyl group, trifluoromethyl group, and the above groups may also be further substituted with substituents acceptable in the art.

In the present disclosure, the “number of ring atoms” refers to the number of atoms among atoms constituting the ring itself of a structural compound obtained by atomic bonding to form a ring, and is, for example, a monocyclic compound, a fused ring compound, a crosslinked compound, a carbocyclic compound, or a heterocyclic compound. When the ring is substituted with a substituent, the atoms contained in the substituent are not included in the ring-forming atoms. The same applies to the “number of ring atoms” described below unless otherwise specified. For example, the number of ring atoms of the benzene ring is 6, the number of ring atoms of the naphthalene ring is 10, and the number of ring atoms of the thienyl group is 5.

In the present disclosure, “aryl group or aromatic group” refers to an aromatic hydrocarbon group derived by removing one hydrogen atom on the basis of an aromatic ring compound, and may be a monocyclic aryl group, a fused ring aryl group, or a polycyclic aryl group, and at least one of the polycyclic rings is an aromatic ring system. For example, “substituted or unsubstituted aryl group having 6 to 40 ring atoms” refers to an aryl group having 6 to 40 ring atoms, in at least one embodiment a substituted or unsubstituted aryl group having 6 to 30 ring atoms, in another embodiment a substituted or unsubstituted aryl group having 6 to 18 ring atoms, in another embodiment a substituted or unsubstituted aryl group having 6 to 14 ring atoms, and the aryl group is optionally further substituted. Suitable examples include, but not limited to, phenyl, biphenyl, terphenyl, naphthyl, anthracenyl, phenanthrenyl, fluoranthenyl, triphenylene, pyrenyl, perylenyl, tetraphenyl, fluorenyl, perylene, acenaphthenyl, and derivatives thereof. It should be understood that a plurality of aryl groups may also be interrupted by short non-aromatic units (e.g. non-H atoms such as C atoms, N atoms or O atoms in a molar ratio of less than 10%), in particular acenaphthene, fluorene (such as 9,9-diarylfluorene), triarylamine, diaryl ether systems should also be included in the definition of aryl groups.

In the present disclosure, “heteroaryl, heteroaryl group or heteroaromatic group” refers to at least one carbon atom on the basis of the aryl group replaced by a non-carbon atom, which may be an N atom, O atom, S atom, etc. For example, “substituted or unsubstituted heteroaryl group having 5 to 40 ring atoms” refers to a heteroaryl group having 5 to 40 ring atoms, in at least one embodiment a substituted or unsubstituted heteroaryl group having 6 to 30 ring atoms, in another embodiment a substituted or unsubstituted heteroaryl group having 6 to 18 ring atoms, in another embodiment a substituted or unsubstituted heteroaryl group having 6 to 14 ring atoms, and the heteroaryl group is optionally further substituted. Suitable examples may include, but not limited to, thienyl, furyl, pyrrolyl, imidazolyl, oxadiazolyl, triazolyl, imidazolyl, pyridyl, bipyridyl, pyrimidinyl, triazinyl, acridinyl, pyridazinyl, pyrazinyl, quinolinyl, isoquinolinyl, quinazolinyl, isoquinolinyl, quinoxalinyl, phthalazinyl, pyridopyrimidinyl, pyridopyrazinyl, benzothienyl, benzofuryl, indolyl, pyridopyrimidinyl, benzofuryl, indolyl, pyridopyrimidinyl, pyridopyrimidinyl, benzofuryl, indolyl, pyridopyrimidinyl, thienopyrrolyl, thienofuryl, carbazolyl, and derivatives thereof.

In the present disclosure, “alkyl” may represent a linear alkyl group, a branched alkyl group, and/or a cyclic alkyl group. The carbon number of the alkyl group may be 1 to 50, 1 to 30, 1 to 20, 1 to 10, or 1 to 6. A phrase including the term, for example, “C₁₋₉ alkyl” refers to an alkyl group having 1 to 9 carbon atoms, which at each occurrence may be independently of each other C₁ alkyl, C₂ alkyl, C₃ alkyl, C₄ alkyl, C₅ alkyl, C₆ alkyl, C₇ alkyl, C₈ alkyl, or C₉ alkyl. Non-limiting examples of alkyl group include methyl, ethyl, n-propyl, isopropyl, n-butyl, sec-butyl, tert-butyl, isobutyl, 2-ethylbutyl, 3,3-dimethylbutyl, n-pentyl, isopentyl, neopentyl, tert-pentyl, cyclopentyl, 1-methylpentyl, 3-methylpentyl, 2-ethylpentyl, 4-methyl-2-pentyl, n-hexyl, 1-methythexyl, 2-ethylhexyl, 2-butylhexyl, cyclohexyl, 4-methylcyclohexyl, 4-tert-butylcyclohexyl, n-heptyl, 1-methylheptyl, 2,2-dimethylheptyl, 2-ethylheptyl, 2-butylheptyl, N-octyl, T-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldodecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-hexadecyl, n-docosyl, n-tridecyl, n-tetracosyl, n-pentadecyl, n-hexadecyl, n-octadecyl, n-nonadecyl, n-tridecyl, and the like.

In the present disclosure, “amino” refers to a derivative of an amine having the structural feature of formula —N(X)₂, where each “X” is independently H, a substituted or unsubstituted alkyl group, a substituted or unsubstituted cycloalkyl group, a substituted or unsubstituted heterocyclyl group, and the like. Non-limiting types of amino groups include —NH₂, —N(alkyl)₂, —NH(alkyl), —N(cycloalkyl)₂, —NH(cycloalkyl), —N(heterocyclyl)₂, —NH(heterocyclyl), —N(aryl)₂, —NH(aryl), —N(alkyl)(aryl), —N(alkyl)(heterocyclyl), —N(cycloalkyl)(heterocyclyl), —N(aryl)(heteroaryl), —N(alkyl)(heteroaryl), and the like.

In the present disclosure, when the attachment site is not specified in the group, it means that an optional attachable site in the group is used as the attachment site.

In the present disclosure, a single bond to which a substituent is attached runs through the corresponding ring, indicating that the substituent may be attached to an optional position of the ring, for example, R in

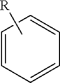

may be attached to any substitutable position of the benzene ring.

In the present disclosure, the organic alkali refers to an amine compound and a nitrogen-containing heterocyclic compound, which in turn refers to a C₈-C₂₂ aliphatic amine and an aromatic amine, and the aromatic amine refers to a heteroaryl group having 5 to 30 ring atoms, and at least one heteroatom of the aromatic amine is selected from N.

First aspect, embodiments of the present disclosure provide an organic compound including a compound represented by the general formula (1):

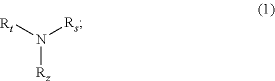

Where, each time R_{z}, Rₛ and Rₜ appear are independently selected from

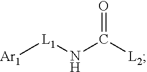

Each time Ar₁ appears is independently selected from an aryl group having 6 to 30 ring atoms that is unsubstituted or substituted with at least one R, or a heteroaryl group having 5 to 30 ring atoms that is unsubstituted or substituted with at least one R, or any combination of these groups; a heteroatom of the heteroaryl group is selected from one or more of N, S, O, P, and Si, and a number of the heteroatom of the heteroaryl group is selected from 1 to 20;

Each time L₁ appears is independently selected from a single bond, a C₁-C₃₀ aliphatic chain hydrocarbon group unsubstituted or substituted with at least one R, a hydroxyl group, an aldehyde group, a sulfhydryl group, an ether group, an ester group, an amino group, a carbonyl group, a nitro group, or a halogen;

Each time L₂ appears is independently selected from a C₁-C₃₀ aliphatic chain hydrocarbon group unsubstituted or substituted with at least one R, hydroxyl group, an aldehyde group, a sulfhydryl group, an ether group, an ester group, an amino group, a carbonyl group, or a nitro group;

Each time R appears is independently selected from deuterium, a halogen, a hydroxyl group, a carboxyl group, a nitro group, a sulfonyl group, a sulfhydryl group, a cyano group, an aldehyde group, an amide group, or any combination of these groups.

In the compound represented by the general formula (1) provided in the present disclosure, the organic compound is capable of chelating uncoordinated metals due to the amide group and the L₂ group, thereby, when the organic compound is applied to an optoelectronic device, the organic compound may act as a modification layer between the hole functional layer and the quantum dot layer to modify and chelate the uncoordinated metal on the surface of the hole functional layer and the surface of the quantum dot layer, thereby improving the surface defects of the film, improving the carrier transport efficiency, and further improving the luminous efficiency of the optoelectronic device and prolonging the lifetime of the optoelectronic device.

In some embodiments, each time Ar₁ appears is independently selected from the following groups:

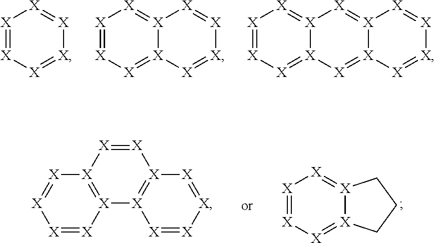

Where each time X appears is independently selected from CR₁ or NR₁; each time R₁ appears is independently selected from hydrogen, deuterium, a halogen, a hydroxyl group, a carboxyl group, a nitro group, a sulfonyl group, a sulfhydryl group, a cyano group, an aldehyde group, an amide group, a C₁-C₃₀ aliphatic chain hydrocarbon group unsubstituted or substituted with at least one R, a C₁-C₃₀ aliphatic chain hydrocarbon oxy group unsubstituted or substituted with at least one R, a C₃-C₃₀ aliphatic cyclohydrocarbon group unsubstituted or substituted with at least one R, a C₃-C₃₀ aliphatic heterocyclohydrocarbon group unsubstituted or substituted with at least one R, an aryl group having 6 to 30 ring atoms that is unsubstituted or substituted with at least one R, an aryloxy group having 6 to 30 ring atoms that is unsubstituted or substituted with at least one R; a heteroaryl group having 5 to 30 ring atoms unsubstituted or substituted with at least one R, or a heteroaryloxy group having 5 to 30 ring atoms unsubstituted or substituted with at least one R, or any combination of these groups; heteroatoms in the aliphatic heterocyclic hydrocarbyl group, the heteroaryl group, and the heteroaryloxy group are each independently selected from one or more of N, S, O, P, and Si, and numbers of heteroatoms in the aliphatic heterocyclic hydrocarbyl group, the heteroaryl group, and the heteroaryloxy group are each independently selected from 1 to 20;

Each time Y appears is independently selected from S, N, O, a C₁-C₃₀ aliphatic chain hydrocarbon group unsubstituted or substituted with at least one R, a C₃-C₃₀ aliphatic cyclic hydrocarbon group unsubstituted or substituted with at least one R, or a C₃-C₃₀ aliphatic heterocyclic hydrocarbon group unsubstituted or substituted with at least one R.

In some embodiments, furthermore, the general formula (1) is selected from general formula (2-1) or general formula (2-2):

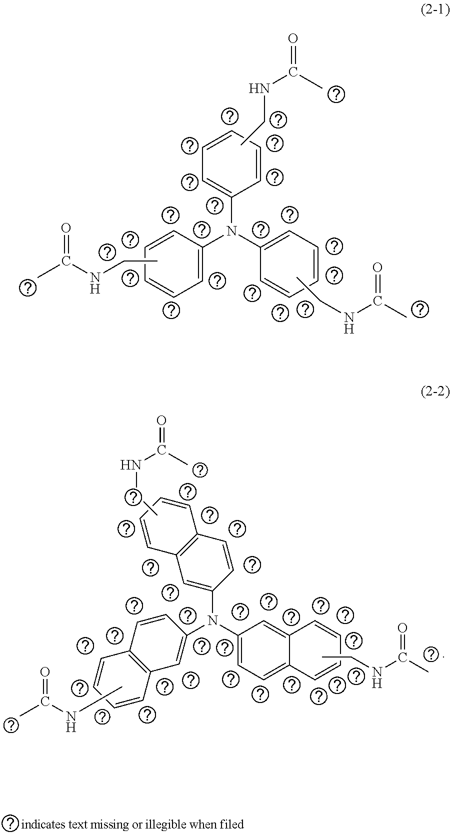

In some embodiments, each time Ar₁ appears is independently selected from an aryl group having 6 to 10 ring atoms that is unsubstituted or substituted with at least one R;

Each time L₁ appears is independently selected from a single bond, a C₁-C₈ linear hydrocarbon group unsubstituted or substituted with at least one R, a C₃-C₈ branched hydrocarbon group unsubstituted or substituted with at least one R;

Each time L₂ appears is independently selected from a C₁-C₃₀ halogenated hydrocarbon group.

In some embodiments, the organic compound is selected from any of the following structures:

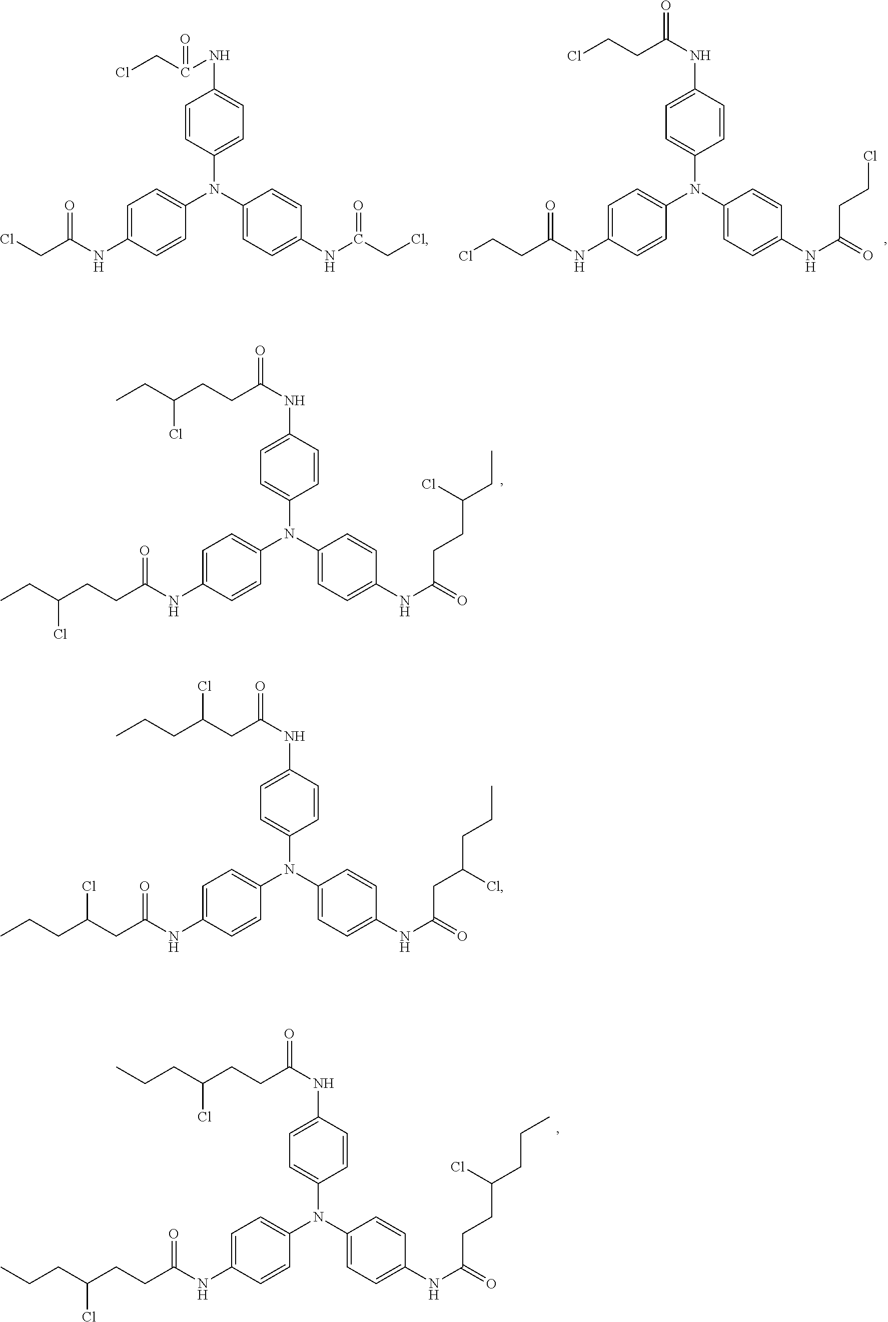 [SMILES: O=C(CCl)Nc1ccc(N(c2ccc(NC(=O)CCl)cc2)c2ccc(NC(=O)CCl)cc2)cc1 ; O=C(CCCl)Nc1ccc(N(c2ccc(NC(=O)CCCl)cc2)c2ccc(NC(=O)CCCl)cc2)cc1 ; CCC(Cl)CCC(=O)Nc1ccc(N(c2ccc(NC(=O)CCC(Cl)CC)cc2)c2ccc(NC(=O)CCC(Cl)CC)cc2)cc1 ; CCCC(Cl)CC(=O)Nc1ccc(N(c2ccc(NC(=O)CC(Cl)CCC)cc2)c2ccc(NC(=O)CC(Cl)CCC)cc2)cc1 ; CCCC(Cl)CCC(=O)Nc1ccc(N(c2ccc(NC(=O)CCC(Cl)CCC)cc2)c2ccc(NC(=O)CCC(Cl)CCC)cc2)cc1]

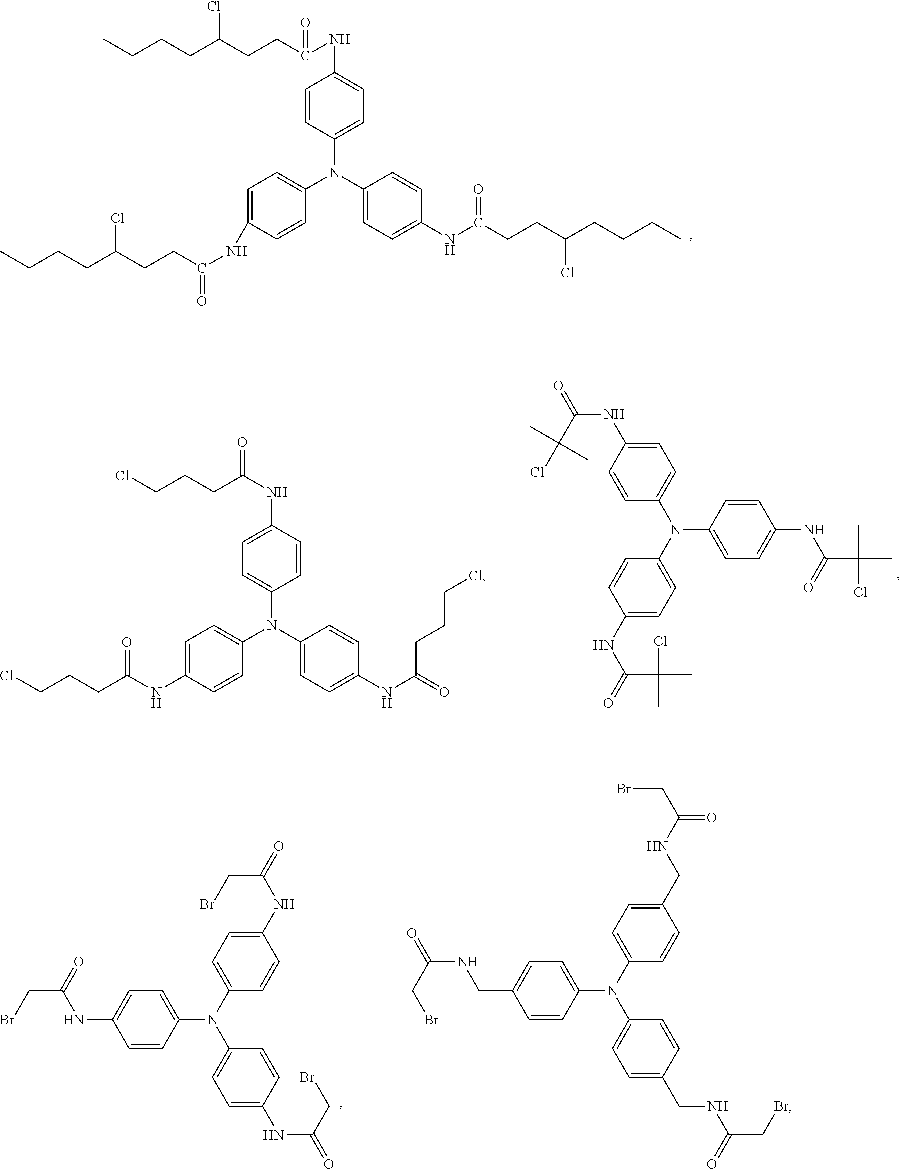

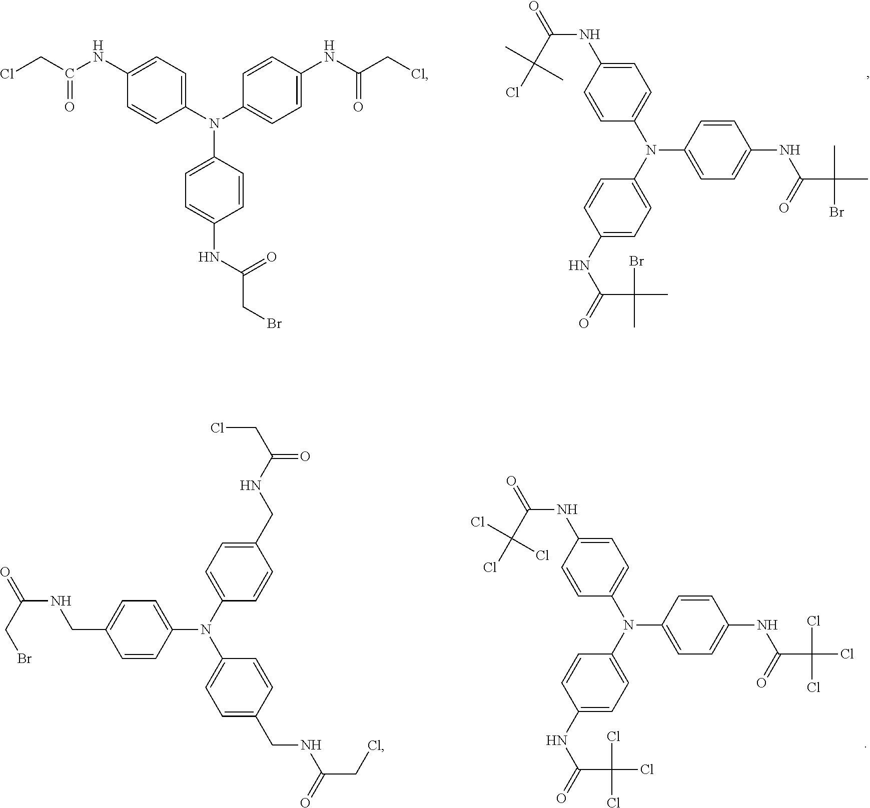 [SMILES: O=C(CCl)Nc1ccc(N(c2ccc(NC(=O)CCl)cc2)c2ccc(NC(=O)CBr)cc2)cc1 ; CC(C)(Cl)C(=O)Nc1ccc(N(c2ccc(NC(=O)C(C)(C)Br)cc2)c2ccc(NC(=O)C(C)(C)Br)cc2)cc1 ; O=C(CCl)NCc1ccc(N(c2ccc(CNC(=O)CCl)cc2)c2ccc(CNC(=O)CBr)cc2)cc1 ; O=C(Nc1ccc(N(c2ccc(NC(=O)C(Cl)(Cl)Cl)cc2)c2ccc(NC(=O)C(Cl)(Cl)Cl)cc2)cc1)C(Cl)(Cl)Cl]

Second aspect, embodiments of the present disclosure further provide a method of synthesizing an organic compound, the method includes the following steps:

Mixing a first compound and a second compound in a first solvent to react to obtain an organic compound.

A structure of the first compound is represented by the general formula (I):

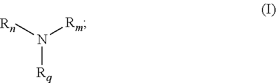

Where each time Rₘ, Rₙ and R_{q} appear are each independently selected from

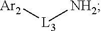

Each time Ar₂ appears is independently selected from an aryl group having 6 to 30 ring atoms that is unsubstituted or substituted with at least one R, or a heteroaryl group having 5 to 30 ring atoms that is unsubstituted or substituted with at least one R, or any combination of these groups; a heteroatom of the heteroaryl group is selected from one or more of N, S, O, P, and Si, and a number of the heteroatom in the heteroaryl group is selected from 1 to 20;

Each time L₃ appears is independently selected from a single bond, a C₁-C₃₀ aliphatic chain hydrocarbon group unsubstituted or substituted with at least one R, a hydroxyl group, an aldehyde group, a sulfhydryl group, an ether group, an ester group, an amino group, a carbonyl group, a nitro group, or a halogen;

Each time R appears is independently selected from deuterium, a halogen, a hydroxyl group, a carboxyl group, a nitro group, a sulfonyl group, a sulfhydryl group, a cyano group, an aldehyde group, an amide group, or any combination of these groups.

A structure of the second compound is represented by the general formula (II):

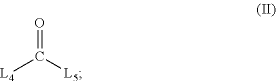

Where L₄ is selected from a C₁-C₃₀ aliphatic chain hydrocarbon group unsubstituted or substituted with at least one R, a hydroxyl group, an aldehyde group, a sulfhydryl group, an ether group, an ester group, an amino group, a carbonyl group, or a nitro group;

L₅ is selected from a halogen;

Each time R appears is independently selected from deuterium, a halogen, a hydroxyl group, a carboxyl group, a nitro group, a sulfonyl group, a sulfhydryl group, a cyano group, an aldehyde group, an amide group, or any combination of these groups.

In some embodiments, each time Ar₂ appears is independently selected from the following groups:

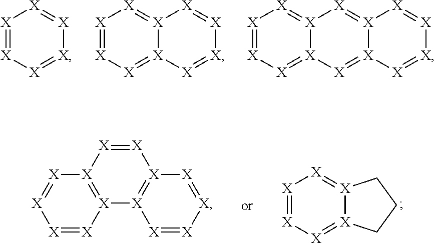

Where each time X appears is independently selected from CR₁ or NR₁; each time R₁ appears is independently selected from hydrogen, deuterium, a halogen, a hydroxyl group, a carboxyl group, a nitro group, a sulfonyl group, a sulfhydryl group, a cyano group, an aldehyde group, an amide group, a C₁-C₃₀ aliphatic chain hydrocarbon group unsubstituted or substituted with at least one R, a C₁-C₃₀ aliphatic chain hydrocarbon oxy group unsubstituted or substituted with at least one R, a C₃-C₃₀ aliphatic cyclohydrocarbon group unsubstituted or substituted with at least one R, a C₃-C₃₀ aliphatic heterocyclohydrocarbon group unsubstituted or substituted with at least one R, an aryl group having 6 to 30 ring atoms that is unsubstituted or substituted with at least one R, an aryloxy group having 6 to 30 ring atoms that is unsubstituted or substituted with at least one R, a heteroaryl group having 5 to 30 ring atoms that is unsubstituted or substituted with at least one R, or a heteroaryloxy group having 5 to 30 ring atoms that is unsubstituted or substituted with at least one R, or any combination of these groups; heteroatoms in the aliphatic heterocyclic hydrocarbyl group, the heteroaryl group, and the heteroaryloxy group are each independently selected from one or more of N, S, O, P, and Si, and numbers of heteroatoms in the aliphatic heterocyclic hydrocarbyl group, the heteroaryl group, and the heteroaryloxy group are each independently selected from 1 to 20;

Each time Y appears is independently selected from S, N, O, a C₁-C₃₀ aliphatic chain hydrocarbon group unsubstituted or substituted with at least one R, a C₃-C₃₀ aliphatic cyclic hydrocarbon group unsubstituted or substituted with at least one R, or a C₃-C₃₀ aliphatic heterocyclic hydrocarbon group unsubstituted or substituted with at least one R.

A third compound is further provided, and the third compound is mixed with the first compound and the second compound in the first solvent. The third compound includes an organic alkali.

The first solvent includes an organic solvent.

In some embodiments, furthermore, the general formula (I) is selected from the general formula (I-1) or the general formula (1-2):

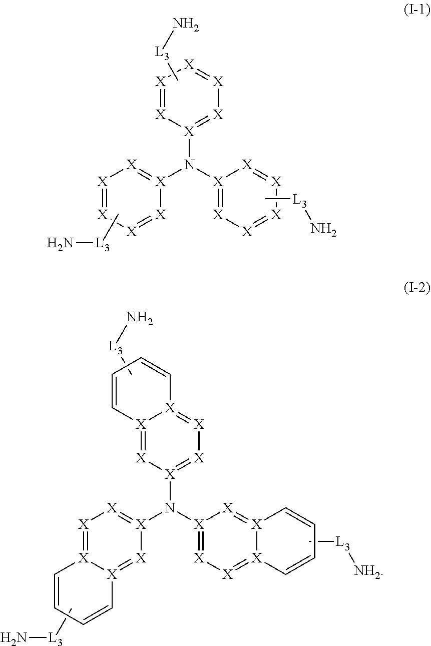

In some embodiments, each time Ar₂ appears is independently selected from an aryl group having 6 to 10 ring atoms that is unsubstituted or substituted with at least one R;

Each time L₃ appears is independently selected from a single bond, a C₁-C₈ linear hydrocarbon group unsubstituted or substituted with at least one R, or a C₃-C₈ branched hydrocarbon group unsubstituted or substituted with at least one R;

Each time L₄ appears is independently selected from a C₁-C₃₀ halogenated hydrocarbon group.

In some embodiments, the third compound is selected from NR₁R₂R₃, where each time Ri, R₂, and R₃ appear are each independently selected from hydrogen, deuterium, or a C₁-C₃₀ aliphatic chain hydrocarbon group unsubstituted or substituted with at least one R. In at least one embodiment, the third compound is selected from tertiary amines, such as triethylamine, in order to avoid by-products generated after the end of the reaction.

In some embodiments, the step of mixing a first compound and a second compound in a first solvent specifically includes: dissolving a first compound in a first solvent, then adding a third compound, slowly dropping a second compound at 0° C., and stirring and reacting at 25° C.

In some embodiments, before the step of mixing a first compound and a second compound in a first solvent, the method further includes a step of synthesizing the first compound, and the step of synthesizing the first compound includes:
Providing a first precursor having a general formula (III) and a second precursor having a general formula (IV), dissolving the first precursor and the second precursor in a precursor solvent, and heating to obtain a first intermediate product;
Dissolving a fourth compound and the first intermediate product in a second intermediate solvent to obtain the first compound, and the fourth compound is selected from one or more of hydrazine hydrate, borane, diisobutylaluminum hydride, lithium aluminum hydride, sodium borohydride, and potassium borohydride.

The general formula (III) is:

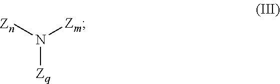

Where each time Zₘ, Zₙ and Z_{q} appear are independently selected from Ar₂-L₃.

The general formula (IV) is:

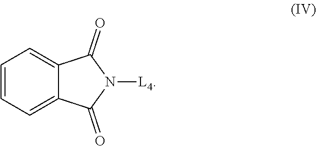

In some embodiments, the step of dissolving the first precursor and the second precursor in a precursor solvent specifically includes: dissolving the first precursor, the second precursor, CuI, a strong alkali and weak acid salt, and

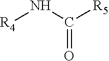

in a precursor solvent; where each time R₄ and R₅ appears are each independently selected from a C₁-C₃₀ aliphatic chain hydrocarbon group unsubstituted or substituted with at least one R.

The first solvent includes dichloromethane.

The precursor solvent is selected from one or more of 1,4-dioxane, dimethyl sulfoxide, toluene, and xylene.

A boiling point of the second intermediate solvent is less than or equal to 100° C. In at least one embodiment, the second intermediate solvent is acetonitrile. The second intermediate solvent having a lower boiling point is selected to facilitate removal under vacuum.

In the embodiments of the present disclosure, the strong alkali and weak acid salt refers to a salt generated by the reaction of a strong alkali and a weak acid, and is alkaline in water. A cation in the strong alkali and weak acid salt is selected from Na⁺, K⁺, Ca²⁺, or Ba²⁺, but is not limited thereto. The anion is selected from carbonate ion, sulfite ion, hydrogen sulfide ion, silicate ion, metaaluminate ion, hypochlorite ion, acetate ion, and the like, but is not limited thereto.

Third aspect, embodiments of the present disclosure further provide an optoelectronic device including a first electrode, a functional layer, and a second electrode stacked in this order. The functional layer includes the organic compound described above, or the organic compound synthesized by the method of synthesizing an organic compound described above.

In some embodiments of the present disclosure, the first electrode and the second electrode may independently include, for example, but not limited to, a doped metal oxide electrode, a composite electrode, a graphene electrode, a carbon nanotube electrode, a metal elemental electrode, or an alloy electrode. A material of the doped metal oxide electrode may include, but not limited to, one or more of indium-doped tin oxide (ITO), fluorine-doped tin oxide (FTO), antimony-doped tin oxide (ATO), aluminum-doped zinc oxide (AZO), gallium-doped zinc oxide (GZO), indium-doped zinc oxide (IZO), magnesium-doped zinc oxide (MZO), aluminum-doped magnesium oxide (AMO), and cadmium-doped zinc oxide. The composite electrode includes AZO/Ag/AZO, AZO/Al/AZO, ITO/Ag/ITO, ITO/Al/ITO, ZnO/Ag/ZnO, ZnO/Al/ZnO, TiO₂/Ag/TiO₂, TiO₂/Al/TiO₂, ZnS/Ag/ZnS, ZnS/Al/ZnS, Ca/Al, LiF/Ca, LiF/Al, BaF₂/Al, CsF/Al, CaCO/Al, or BaF₂/Al. A material of the metal elemental electrode may include, but not limited to, one or more of Ag, Ni, Pt, Au, Ir, Cu, Mo, Al, Ca, Mg, and Ba. The alloy electrode includes, but not limited to, Au:Mg alloy electrode or Ag:Mg alloy electrode.

The functional layer includes a hole functional layer, a modification layer, an excitation layer and an electronic functional layer stacked in this order.

A material of the hole functional layer includes one or more of 4,4′-N,N′-dicarbazolyl-biphenyl, poly [bis(4-phenyl) (2,4,6-trimethylphenyl) amine], N,N′-diphenyl-N,N′-bis(1-naphthyl)-1,1′-biphenyl-4,4′-diamine, N,N′-diphenyl-N,N′-bis(3-methylphenyl)-(1,1′-biphenyl)-4,4′-diamine, poly (N,N′bis(4-butylphenyl)-N,N′-bis(phenyl) benzidine), N,N′-bis(3-methylphenyl)-N,N′-bis(phenyl)-spiro, N,N′-bis(4-(N,N′-diphenyl-amino) phenyl)-N,N′-diphenylbenzidine, 4,4′,4′-tris(N-carbazolyl)-triphenylamine, 4,4′,4′-tris(N-3-methylphenyl-N-phenylamino) triphenylamine, poly [(9,9′-dioctylfluorene-2,7-diyl)-co-(4,4′-(N-(4-sec-butylphenyl) diphenylamine)], poly (N-vinylcarbazole) and derivatives thereof, N,N′-bis(1-naphthyl)-N,N′-diphenyl-1,1′-biphenyl-4-4′-diamine, spiro NPB, poly (phenylenevinylene), poly [2-methoxy-5-(2-ethylhexyloxy)-1,4-phenylenevinylene], poly [2-methoxy-5-(3′,7′-dimethyloctoxy)-1,4-phenylenevinylene], 2,2′,7,7′-tetrakis [N,N-bis(4-methoxyphenyl) amino]-9,9′-spirobifluorene, 4,4′-cyclohexyl bis[N,N-bis (4-methylphenyl) aniline], 1,3-bis(carbazol-9-yl) benzene, polyaniline, polypyrrole, poly (p) phenylenevinylidene, aromatic tertiary amine, polynuclear aromatic tertiary amine, 4,4′-bis(p-carbazolyl)-1,1′-biphenyl compound, N,N,N′,N′-tetraarylbenzidine, PEDOT:PSS and derivatives thereof, polymethacrylates and derivatives thereof, poly (9,9-octylfluorene) and derivatives thereof, poly (spirofluorene) and derivatives thereof, 2,3,6,7,10,11-hexacyano-1,4,5,8,9,12-hexaazabenzophenanthrene, PEDOT, PEDOT:PSS doped with s-MoO₃ derivatives, 4,4′,4′-tris(N-3-methylphenyl-N-phenylamino) triphenylamine, tetracyanoquinone dimethane, doped graphene, undoped graphene, C60, copper phthalocyanine, a second doped metal oxide particle, a second undoped metal oxide particle, a metal sulphide, and a metal nitride.

The modification layer includes the organic compound described above, or the organic compound synthesized by the method of synthesizing an organic compound described above.

A material of the excitation layer includes one or more of a single structure quantum dot, a core-shell structure quantum dot, and a perovskite-type semiconductor material.

A material of the single structure quantum dot, a material of the core of the core-shell structure quantum dot, and a material of the shell of the core-shell structure quantum dot may include, but not limited to, one or more of a Group II-VI compound, a Group IV-VI compound, a Group III-V compound, and a Group I-III-VI compound, respectively. The Group II-VI compound may include, but not limited to, one or more of CdS, CdSe, CdTe, ZnS, ZnSe, ZnTe, ZnO, HgS, HgSe, HgTe, CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, CdHgTe, HgZnS, HgZnSe, HgZnTe, CdZnSeS, CdZnSeTe, CdZnSTe, CdHgSeTe, CdHgSTe, HgZnSeS, HgZnSeTe, and HgZnSTe. The Group IV-VI compound may include, but not limited to, one or more of SnS, SnSe, SnTe, PbS, PbSe, PbTe, SnSeS, SnSeTe, SnSTe, PbSeS, PbSeTe, PbSTe, SnPbS, SnPbSe, SnPbTe, SnPbSSe, SnPbSeTe, and SnPbSTe. The Group III-V compound may include, but not limited to one or more of GaN, GaP, GaAs, GaSb, AlN, AlP, AlAs, AlSb, InN, InP, InAs, InSb, GaNP, GaNAs, GaNSb, GaPAs, GaPSb, AlNP, AlNAs, AlNSb, AlPAs, AlPSb, InNP, InNAs, InNSb, InPAs, InPSb, GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, and InAlPSb. The Group 1-III-VI compound may include, but not limited to, one or more of CuInS₂, CuInSe₂, and AgInS₂.

The perovskite-type semiconductor material may include, but not limited to, a doped or undoped inorganic perovskite-type semiconductor, or an organic-inorganic hybrid perovskite-type semiconductor. A general structural formula of the inorganic perovskite-type semiconductor is AMX₃, where A is a Cs⁺ ion, M is a divalent metal cation including one or more of Pb²⁺, Sn²⁺, Cu²⁺, Ni²⁺, Cd²⁺, Cr²⁺, Mn²⁺, Co²⁺, Fe²⁺, Ge²⁺, Yb²⁺, and Eu²⁺, and X is a halogen anion including one or more of Cl⁻, Br⁻, and I⁻. A general structural formula of the organic-inorganic hybrid perovskite-type semiconductor is BMX₃, where B is an organic amine cation including CH₃(CH₂)ₙ₋₂NH₃⁺ or [NH₃(CH₂)ₙNH₃]²⁺, where n≥2, M is a divalent metal cation including one or more of Pb²⁺, Sn²⁺, Cu²⁺, Ni²⁺, Cd²⁺, Cr²⁺, Mn²⁺, Co²⁺, Fe²⁺, Ge²⁺, Yb²⁺, and Eu²⁺, and X is a halogen anion including one or more of Cl⁻, Br, and I.

A material of the electronic functional layer includes one or more of an inorganic electronic functional material and an organic electronic functional material. The inorganic electronic functional material includes one or more of a first doped metal oxide particle, a first undoped metal oxide particle, a Group IIB-VIA semiconductor material, a Group IIIA-VA semiconductor material, and a Group IB-IIIA-VIA semiconductor material. The organic electronic functional material includes one or more of a quinoxaline compound, an imidazole compound, a triazine compound, a fluorene-containing compound, a hydroxyquinoline compound, and a fullerene derivative.

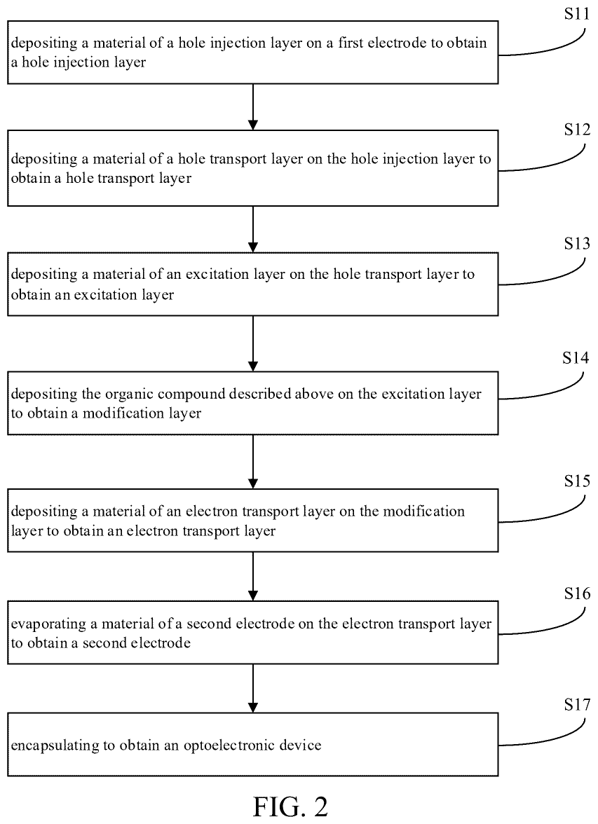

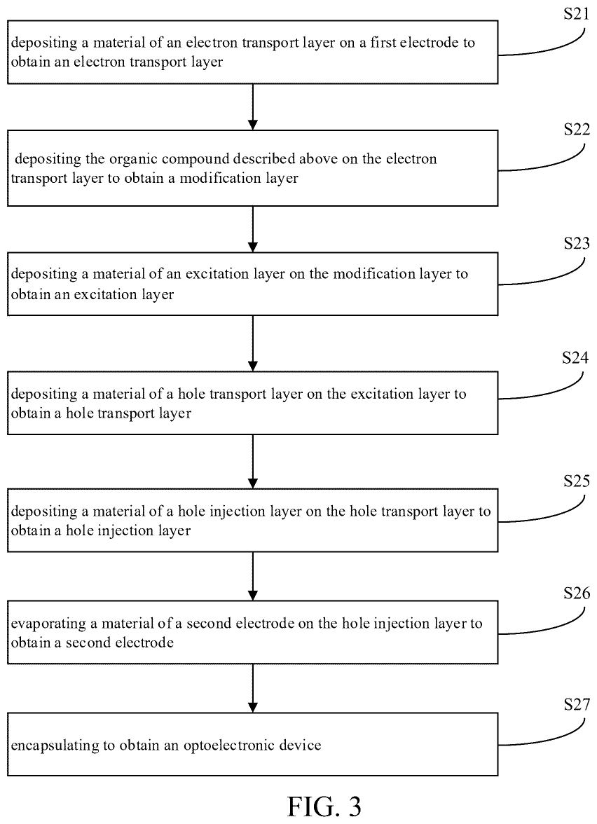

Fourth aspect, referring to FIG. 2, embodiments of the present disclosure further provide a method for preparing an optoelectronic device, and the method includes the following steps:
S11: depositing a material of a hole injection layer on a first electrode to obtain a hole injection layer;
S12: depositing a material of a hole transport layer on the hole injection layer to obtain a hole transport layer;
S13: depositing a material of an excitation layer on the hole transport layer to obtain an excitation layer;
S14: depositing the organic compound described above on the excitation layer to obtain a modification layer;
S15: depositing a material of an electron transport layer on the modification layer to obtain an electron transport layer;
S16: evaporating a material of a second electrode on the electron transport layer to obtain a second electrode;
S17: encapsulating to obtain an optoelectronic device.
In some embodiments, referring to FIG. 3, embodiments of the present disclosure further provide another method for preparing an optoelectronic device, and the method includes the following steps:
S21: depositing a material of an electron transport layer on a first electrode to obtain an electron transport layer;
S22: depositing the organic compound described above on the electron transport layer to obtain a modification layer;
S23: depositing a material of an excitation layer on the modification layer to obtain an excitation layer;
S24: depositing a material of a hole transport layer on the excitation layer to obtain a hole transport layer;
S25: depositing a material of a hole injection layer on the hole transport layer to obtain a hole injection layer;
S26: evaporating a material of a second electrode on the hole injection layer to obtain a second electrode;
S27: encapsulating to obtain an optoelectronic device.

The depositing method may be realized by technical means well known in the art.

The depositing method of the excitation layer may be a chemical vapor deposition method, a continuous ion layer adsorption and reaction method, an anodic oxidation method, a co-precipitation method, and a solution processing method.

The depositing method of the hole transport layer may be a physical coating method or a solution processing method. The physical coating method may be a thermal evaporation coating method, an electron beam evaporation coating method, a magnetron sputtering method, a multi-arc ion coating method, a physical vapor deposition method, an atomic layer deposition method, or a pulsed laser deposition method. The solution processing method may be a spin coating method, a printing method, an inkjet printing method, a blade coating method, a printing method, a dipping and pulling method, an immersion method, a spray coating method, a roll coating method, a casting method, a slit coating method, or a strip coating method.

The depositing method of the electron transport layer may be a chemical vapor deposition method, a continuous ion layer adsorption and reaction method, an anodic oxidation method, a co-precipitation method, or a solution processing method.

When the material of the hole injection layer is an organic molecular compound, all the above-mentioned methods may be employed to form the hole injection layer. When the material of the hole injection layer is an inorganic molecular compound, a solution processing method may be employed to form the hole injection layer.

Specific processing methods and processing conditions may be referred to common methods in the art, and will not be described herein.

Fifth aspect, embodiments of the present disclosure further provide a display device including the optoelectronic device describe above.

The display device may be any electronic product having a display function The electronic product may include, but not limited to, a smartphone, a tablet computer, a laptop computer, a digital camera, a digital video camera, a smart wearable device, a smart weighing electronic scale, a vehicle-mounted display, a television, or an electronic book reader. The smart wearable device may be, for example, a smart bracelet, a smart watch, a Virtual Reality (VR) helmet, and the like.

### Example 1

This Example provides an organic compound and a method of synthesising the organic compound, and the method includes the following steps:
S1: mixing and dissolving 9.83 g of tris-(4-bromophenyl) amine, 1 g of phthalimide, 0.387 g of CuI, 2.16 g of K₃PO₄ and 0.3 mL of N, N-dimethylacetamide in 20 ml of dioxane, refluxing at 110° C. for 24 h, drying to obtain a powder after the reaction is completed, and purifying by silica gel column chromatography with dichloromethane and petroleum ether with a volume ratio of 2.5:1 to obtain the primary product;
S2: slowly dropping 1.6 mL of hydrazine hydrate with a mass fraction of 35% into 60 mL of acetonitrile, further adding 408.5 mg of the primary product, stirring at 25° C. for 2 h, removing the acetonitrile in vacuo to obtain a crude product, further purifying the crude product by silica gel column chromatography with CH₂C₁₂ as an eluent to obtain an intermediate product;
S3: dissolving 0.112 mg of the intermediate product in 5 mL of CH₂C₁₂, and adding 36 μL of triethylamine, then slowly adding 20 μL of chloroacetyl chloride dropwise at 0° C., and stirring at 25° C. for 15 min; after the reaction was finished, removing the CH₂C₁₂ in vacuum to obtain a product need to be purified, further purifying the product need to be purified by silica gel column chromatography with CH₂C₁₂ as an eluent to obtain a crude substance, dissolving the crude substance in CH₂C₁₂, further recrystallizing in n-hexane, then precipitating and filtering, and then washing with n-hexane to obtain an organic compound M1.

The organic compound M1 is:

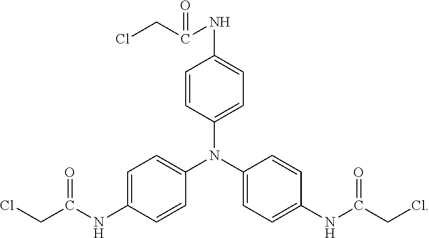 [SMILES: O=C(CCl)Nc1ccc(N(c2ccc(NC(=O)CCl)cc2)c2ccc(NC(=O)CCl)cc2)cc1]

The molecular formula of the organic compound M1 is C₂₄H₂₁N₄O₃Cl₃. The hydrogen nuclear magnetic resonance spectrum parameter of the organic compound M1 is: ¹H NMR (500 MHz, Chloroform-d) δ 9.18 (s, 3H), 7.83-7.77 (m, 6H), 7.24-7.19 (m, 6H), 4.21 (s, 6H).

### Example 2

This Example provides an organic compound and a method of synthesising the organic compound, and the method is different from that of Example 1 in that: the 1,4-dioxane in step S1 was replaced with DMSO, the chloroacetyl chloride in step S3 was replaced with 3-chloropropionyl chloride, and an organic compound M2 was obtained.

The organic compound M2 is:

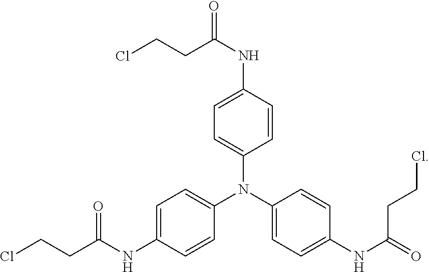 [SMILES: O=C(CCCl)Nc1ccc(N(c2ccc(NC(=O)CCCl)cc2)c2ccc(NC(=O)CCCl)cc2)cc1]

The molecular formula of the organic compound M2 is C₂₇H₂₇N₄O₃Cl₃. The nuclear magnetic hydrogen spectrum parameter of organic compound M2 is: ¹H NMR (500 MHz, Chloroform-d) δ 8.87 (s, 3H), 7.80-7.74 (m, 6H), 7.24-7.19 (m, 6H), 3.86 (t, J=3.1 Hz, 6H), 2.81 (t, J=3.1 Hz, 6H).

### Example 3

This Example provides an organic compound and a method of synthesising the organic compound, and the method is different from Example 1 in that: the hydrazine hydrate in step S2 was replaced with borane, the chloroacetyl chloride in step S3 was replaced with 3-bromopropionyl chloride, the triethylamine in step S3 was replaced with tripropylamine, and an organic compound M2 was obtained.

### Example 4

This Example provides an organic compound and method of synthesising the organic compound, and the method is different from Example 1 in that: the hydrazine hydrate in step S2 was replaced with sodium borohydride, the chloroacetyl chloride in step S3 was replaced with 4-chloroheptanoyl chloride, the triethylamine in step S3 was replaced with tri-n-butylamine, and an organic compound M3 was obtained.

The organic compound M3 is:

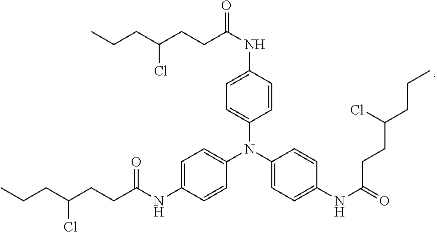 [SMILES: CCCC(Cl)CCC(=O)Nc1ccc(N(c2ccc(NC(=O)CCC(Cl)CCC)cc2)c2ccc(NC(=O)CCC(Cl)CCC)cc2)cc1]

The molecular formula of the organic compound M3 is C₃₉H₅₁N₄O₃Cl₃ The nuclear magnetic hydrogen spectrum parameter of the organic compound M3 is: ¹H NMR (500 MHz, Chloroform-d) δ 8.57 (s, 3H), 7.77-7.73 (m, 6H), 7.24-7.19 (m, 6H), 3.90 (p, J=6.1 Hz, 3H), 2.57-2.49 (m, 6H), 1.92-1.83 (m, 12H), 1.47 (dqt, J=13.0, 7.7, 5.3 Hz, 6H), 0.93 (t, J=7.6 Hz, 9H).

### Example 5

This Example provides an organic compound and method of synthesising the organic compound, and the method is different from that of Example 1 in that: the chloroacetyl chloride in step S3 was replaced with 4-chlorobutyryl chloride, and an organic compound M4 was obtained.

The organic compound M4 is:

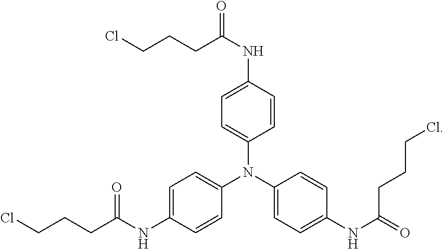 [SMILES: O=C(CCCCl)Nc1ccc(N(c2ccc(NC(=O)CCCCl)cc2)c2ccc(NC(=O)CCCCl)cc2)cc1]

The molecular formula of the organic compound M4 is C₃₀H₃₃N₄O₃Cl₃ The hydrogen nuclear magnetic resonance spectrum parameter of the organic compound M4 is: ¹H NMR (500 MHz, Chloroform-d) δ 8.61 (s, 3H), 7.78-7.72 (m, 6H), 7.24-7.19 (m, 6H), 3.67 (t, J=3.5 Hz, 6H), 2.39 (t, J=7.3 Hz, 6H), 2.17 (tt, J=7.1, 3.5 Hz, 6H).

### Example 6

This Example provides an organic compound and a method of synthesising the organic compound, and the method is different from that of Example 1 in that: the chloroacetyl chloride in step S3 was replaced with 4-bromobutyryl chloride, and organic compound M4 was similarly obtained.

### Example 7

This Example provides an organic compound and a synthesis method thereof, and the method is different from that of Example 1 in that: the chloroacetyl chloride in step S3 was replaced with 2-bromoisobutyryl bromide, and an organic compound M5 was obtained.

The organic compound M5 is:

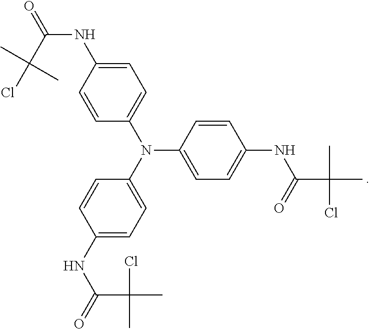 [SMILES: CC(C)(Cl)C(=O)Nc1ccc(N(c2ccc(NC(=O)C(C)(C)Cl)cc2)c2ccc(NC(=O)C(C)(C)Cl)cc2)cc1]

The molecular formula of the organic compound M5 is C₃₀H₂₁N₄O₃Cl₃. The nuclear magnetic hydrogen spectrum parameter of the organic compound M5 is: ¹H NMR (500 MHz, Chloroform-d) δ 7.84-7.78 (m, 6H), 7.48 (s, 3H), 7.24-7.19 (m, 6H), 1.82 (s, 18H).

### Example 8

This Example provides an organic compound and a method of synthesising the organic compound, and the method of synthesising the organic compound is different from Example 1 in that: the chloroacetyl chloride in step S3 was replaced with bromoacetyl bromide, and an organic compound M6 was obtained.

The organic compound M6 is:

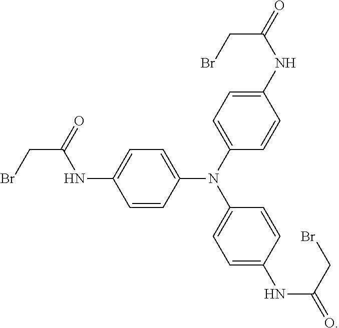 [SMILES: O=C(CBr)Nc1ccc(N(c2ccc(NC(=O)CBr)cc2)c2ccc(NC(=O)CBr)cc2)cc1]

The molecular formula of the organic compound M6 is C₂₄H₂₁N₄O₃Br₃ The hydrogen nuclear magnetic resonance spectrum parameter of the organic compound M6 is: ¹H NMR (500 MHz, Chloroform-d) δ 9.31 (s, 3H), 7.83-7.77 (m, 6H), 7.25-7.19 (m, 6H), 4.02 (s, 6H).

### Example 9

This Example provides an organic compound and a method of synthesising the organic compound, and the method is different from that of Example 1 in that: the tris-(4-bromophenyl) amine in step S1 was replaced with

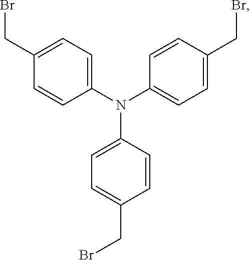 [SMILES: BrCc1ccc(N(c2ccc(CBr)cc2)c2ccc(CBr)cc2)cc1]

Br CAS: 100693-36-5, an organic compound M7 was obtained, and the organic compound M7 is:

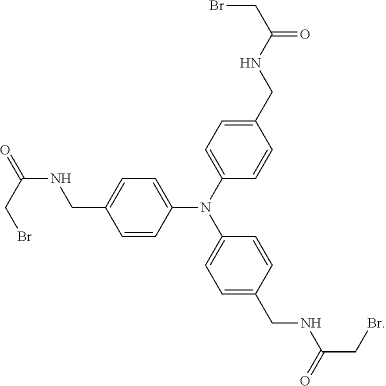 [SMILES: O=C(CBr)NCc1ccc(N(c2ccc(CNC(=O)CBr)cc2)c2ccc(CNC(=O)CBr)cc2)cc1]

The molecular formula of the organic compound M7 is C₂₇H₂₇N₄O₃Br₃. The hydrogen nuclear magnetic resonance spectrum parameter of the organic compound M7 is: ¹H NMR (500 MHz, Chloroform-d) δ 7.38-7.30 (m, 9H), 7.02-6.96 (m, 6H), 4.46 (dt, J=5.5, 0.9 Hz, 6H), 3.83 (s, 6H).

### Example 10

This Example provides an organic compound and a method of synthesising the organic compound, and the method is different from Example 1 in that: 20 μL of chloroacetyl chloride in step S3 was replaced with 5 μL of bromoacetyl bromide and 10 μL of chloroacetyl chloride, and an organic compound M8 was obtained.

The organic compound M8 is:

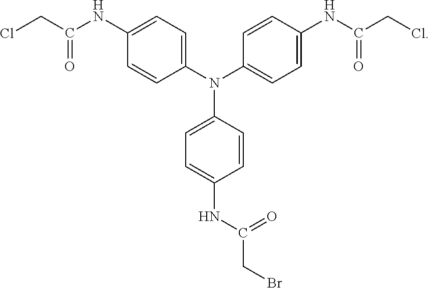 [SMILES: O=C(CCl)Nc1ccc(N(c2ccc(NC(=O)CCl)cc2)c2ccc(NC(=O)CBr)cc2)cc1]

The molecular formula of the organic compound M8 is C₂₄H₂₁N₄O₃BrCl₂. The hydrogen nuclear magnetic resonance spectrum parameters of the organic compound M8 is ¹H NMR (500 MHz, Chloroform-d) δ 9.18 (s, 3H), 7.83-7.77 (m, 6H), 7.24-7.19 (m, 6H), 4.21 (s, 6H).

### Example 11

This Example provides an organic compound and a method of synthesising the organic compound, and the method is different from that of Example 1 in that: 20 μL of chloroacetyl chloride in step S3 was replaced with 5 μL of 2-bromoisobutyryl bromide and 10 μL of 2-bromoisobutyryl chloride, and an organic compound M9 was obtained.

The organic compound M9 is:

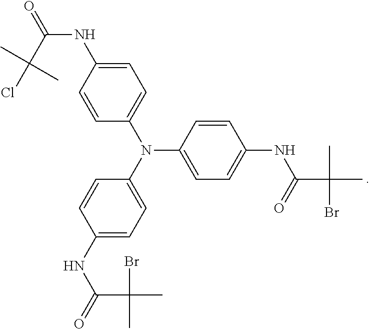 [SMILES: CC(C)(Cl)C(=O)Nc1ccc(N(c2ccc(NC(=O)C(C)(C)Br)cc2)c2ccc(NC(=O)C(C)(C)Br)cc2)cc1]

The molecular formula of the organic compound M9 is C₂₄H₂₁N₄O₃Br₂Cl. The nuclear magnetic hydrogen spectrum parameter of the organic compound M9 is: ¹H NMR (500 MHz, Chloroform-d) δ 7.84-7.78 (m, 6H), 7.48 (s, 3H), 7.24-7.19 (m, 6H), 1.82 (s, 18H).

### Example 12

This Example provides an organic compound and a method of synthesising the organic compound, and the method is different from that of Example 1 in that: the tris-(4-bromophenyl) amine in step S1 was replaced with

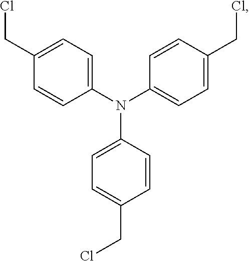 [SMILES: ClCc1ccc(N(c2ccc(CCl)cc2)c2ccc(CCl)cc2)cc1]

CAS: 72393-15-8; 20 μL of chloroacetyl chloride in step S3 was replaced with 5 μL of bromoacetyl bromide and 10 μL of chloroacetyl chloride, and an organic compound M10 was obtained; the organic compound M10 is:

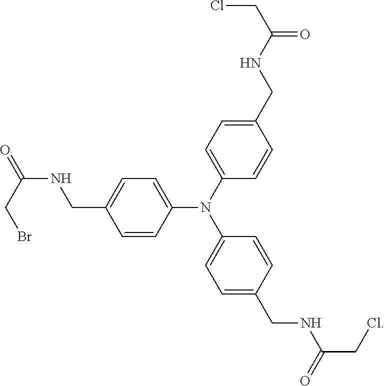 [SMILES: O=C(CCl)NCc1ccc(N(c2ccc(CNC(=O)CCl)cc2)c2ccc(CNC(=O)CBr)cc2)cc1]

The molecular formula of the organic compound M10 is C₂₇H₂₇N₄O₃BrCl₂. The hydrogen nuclear magnetic resonance spectrum parameter of the organic compound M10 is: ¹H NMR (500 MHz, Chloroform-d) δ 7.38-7.30 (m, 9H), 7.02-6.96 (m, 6H), 4.46 (dt, J=5.5, 0.9 Hz, 6H), 3.83 (s, 6H).

### Example 13

This Example provides an organic compound and a method of synthesising the organic compound, and the method is different from that of Example 1 in that: the chloroacetyl chloride in step S3 was replaced with N-chlorotrichloroacetamide, and an organic compound M11 was obtained.

The organic compound M11 is:

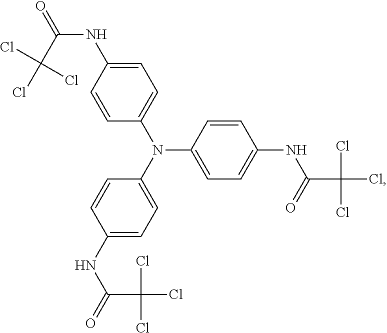 [SMILES: O=C(Nc1ccc(N(c2ccc(NC(=O)C(Cl)(Cl)Cl)cc2)c2ccc(NC(=O)C(Cl)(Cl)Cl)cc2)cc1)C(Cl)(Cl)Cl]

The molecular formula of the organic compound M11 is C₃₀H₂₁N₄O₃Cl₉. The hydrogen nuclear magnetic resonance spectrum parameter of the organic compound M11 is: ¹H NMR (500 MHz, Chloroform-d) δ 7.84-7.78 (m, 6H), 7.48 (s, 3H), 7.24-7.19 (m, 6H), 1.82 (s, 18H).

### Preparation and Characterization of QLED Devices:

Hereinafter, a preparation method for preparing a QLED device using the organic compound of the present disclosure will be described in detail by means of specific device examples.

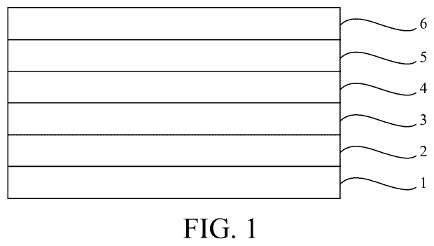

Taking the organic compound M1 as a material of a modification layer as an example, and the prepared QLED device is referred to as “Device Example 1”, the structure of Device Example 1 is shown in FIG. 1, and the preparation method of Device Example 1 includes the following steps:
S01: Providing a substrate of ITO anode with a thickness of 80 nm, ultrasonically cleaning the substrate with acetone and ethanol for 15 min, then cleaning the substrate again with deionized water, then drying the substrate on a heating plate at 150° C. for 10 min, and finally irradiating the substrate with ultraviolet light for 20 min to improve the work function and surface energy of the ITO anode to obtain an anode 1;
S02: spin-coating TFB with a concentration of 8 mg/mL at 3000 rpm for 30 seconds, and then heating at 120° C. for 10 minutes to obtain a hole functional layer 2;
S03: spin-coating CdZnSe quantum dot material with a concentration of 10 mg/mL at 1500 rpm for 30 seconds, and then heating at 100° C. for 5 min to obtain an excitation layer 3;
S04: dissolving the organic compound M1 in 1,1-dichloroethane, dispersing by ultrasonic waves for 10 min to prepare a solution with a concentration of 10 mg/ml, spin-coating the solution at a rotating speed of 4000 rpm to form a film with a thickness of 8 nm, and then UV irradiating for 10 min to obtain a modification layer 4;
S05: spin-coating ZnO-ethanol solution with a concentration of 30 mg/mL at 3000 rpm for 30 seconds, and then heating at 100° C. for 15 minutes to obtain an electron transport layer 5;
S06: Thermal evaporating Ag at a speed of 1 Å/s for 200 seconds and with a vacuum degree less than or equal to 3×10⁻⁴ Pa to obtain a cathode 6 with a thickness of 20 nm, thereby, a QLED-1 was formed.

Furthermore, with reference to the preparation method of Device Example 1, compounds M2 to M11 were used as the materials of the modification layers in QLED devices, respectively, and Device Example 2 to Device Example 11 were prepared accordingly. With reference to the preparation method of Device Example 1, step S4 was omitted, and Device Comparative Example 1 was produced accordingly. It should be understood that in the preparation methods of Device Example 1 to Device Example 8 described above, other experimental conditions were the same except that the materials of the modification layers were different. In the preparation method of the Device Comparative Example 1 described above, the experimental conditions were the same except that step S4 was not included.

In the present disclosure, the characteristics of Device Example 1 to Device Example 11 and Device Comparative Example 1 were characterized, the corresponding luminous efficiency CE (cd/A) and lifetime T95 (1000 nit) of the above devices were measured, and the results are shown in Table 1.

TABLE 1

| | CE(cd/A) | T95 (1000 nit) |
|---|---|---|
| Device Example 1 | 203.7 | 7.31 |
| Device Example 2 | 154.0 | 6.24 |
| Device Example 3 | 144.3 | 6.06 |
| Device Example 4 | 127.6 | 5.53 |
| Device Example 5 | 141.2 | 6.03 |
| Device Example 6 | 195.4 | 7.09 |
| Device Example 7 | 180.5 | 6.88 |
| Device Example 8 | 198.4 | 7.11 |
| Device Example 9 | 140.7 | 5.87 |
| Device Example 10 | 181.4 | 7.01 |
| Device Example 11 | 129.4 | 5.43 |
| Device Comparative | 87.3 | 4.53 |
| Example 1 | | |

It can be seen from Table 1 that Device Example 1 to Device Example 11 have significant improvements in luminous efficiency and lifetime compared with Device Comparative Example 1, a main reason may be that the organic compound in the present disclosure has a better planar structure, and the presence of amide group and L₂ group such as halogenated hydrocarbon may chelate uncoordinated metals on the surfaces of adjacent layers, thereby improving surface defects of the layers, and thus improving the luminous efficiency and lifetime of the device.

The technical solutions provided by the embodiments of the present disclosure are described in detail above. The principles and embodiments of the present disclosure have been described with reference to specific embodiments, and the description of the above embodiments is merely intended to aid in the understanding of the method of the present disclosure and its core idea. At the same time, changes may be made by those skilled in the art to both the specific implementations and the scope of disclosure in accordance with the teachings of the present disclosure. In view of the foregoing, the content of the present specification should not be construed as limiting the disclosure.

What is claimed is:

1. An organic compound, comprising:
a compound represented by the general formula (1): 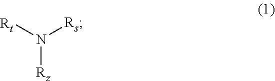
where, each time R_{z}, Rₛ and Rₜ appear are independently selected from 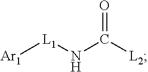
each time Ar₁ appears is independently selected from an aryl group having 6 to 30 ring atoms that is unsubstituted or substituted with at least one R, or a heteroaryl group having 5 to 30 ring atoms that is unsubstituted or substituted with at least one R, or any combination of these groups; a heteroatom of the heteroaryl group is selected from one or more of N, S, O, P, and Si, and a number of the heteroatom of the heteroaryl group is selected from 1 to 20;
each time L₁ appears is independently selected from a single bond, a C₁-C₃₀ aliphatic chain hydrocarbon group unsubstituted or substituted with at least one R, a hydroxyl group, an aldehyde group, a sulfhydryl group, an ether group, an ester group, an amino group, a carbonyl group, a nitro group, or a halogen;
each time L₂ appears is independently selected from a C₁-C₃₀ aliphatic chain hydrocarbon group unsubstituted or substituted with at least one R, hydroxyl group, an aldehyde group, a sulfhydryl group, an ether group, an ester group, an amino group, a carbonyl group, or a nitro group; and
each time R appears is independently selected from deuterium, a halogen, a hydroxyl group, a carboxyl group, a nitro group, a sulfonyl group, a sulfhydryl group, a cyano group, an aldehyde group, an amide group, or any combination of these groups.

2. The organic compound according to claim 1, wherein each time Ar₁ appears is independently selected from the following groups: 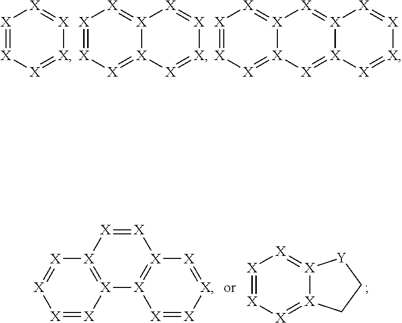
where each time X appears is independently selected from CR₁ or NR₁; each time R₁ appears is independently selected from hydrogen, deuterium, a halogen, a hydroxyl group, a carboxyl group, a nitro group, a sulfonyl group, a sulfhydryl group, a cyano group, an aldehyde group, an amide group, a C₁-C₃₀ aliphatic chain hydrocarbon group unsubstituted or substituted with at least one R, a C₁-C₃₀ aliphatic chain hydrocarbon oxy group unsubstituted or substituted with at least one R, a C₃-C₃₀ aliphatic cyclohydrocarbon group unsubstituted or substituted with at least one R, a C₃-C₃₀ aliphatic heterocyclohydrocarbon group unsubstituted or substituted with at least one R, an aryl group having 6 to 30 ring atoms that is unsubstituted or substituted with at least one R, an aryloxy group having 6 to 30 ring atoms that is unsubstituted or substituted with at least one R; a heteroaryl group having 5 to 30 ring atoms unsubstituted or substituted with at least one R, or heteroaryloxy group having 5 to 30 ring atoms unsubstituted or substituted with at least one R, or any combination of these groups; heteroatoms in the aliphatic heterocyclic hydrocarbyl group, the heteroaryl group, and the heteroaryloxy group are each independently selected from one or more of N, S, O, P, and Si, and numbers of heteroatoms in the aliphatic heterocyclic hydrocarbyl group, the heteroaryl group, and the heteroaryloxy group are each independently selected from 1 to 20; and
each time Y appears is independently selected from S, N, O, a C₁-C₃₀ aliphatic chain hydrocarbon group unsubstituted or substituted with at least one R, a C₃-C₃₀ aliphatic cyclic hydrocarbon group unsubstituted or substituted with at least one R, or a C₃-C₃₀ aliphatic heterocyclic hydrocarbon group unsubstituted or substituted with at least one R.

3. The organic compound according to claim 2, wherein the general formula (1) is selected from general formula (2-1) or general formula (2-2):

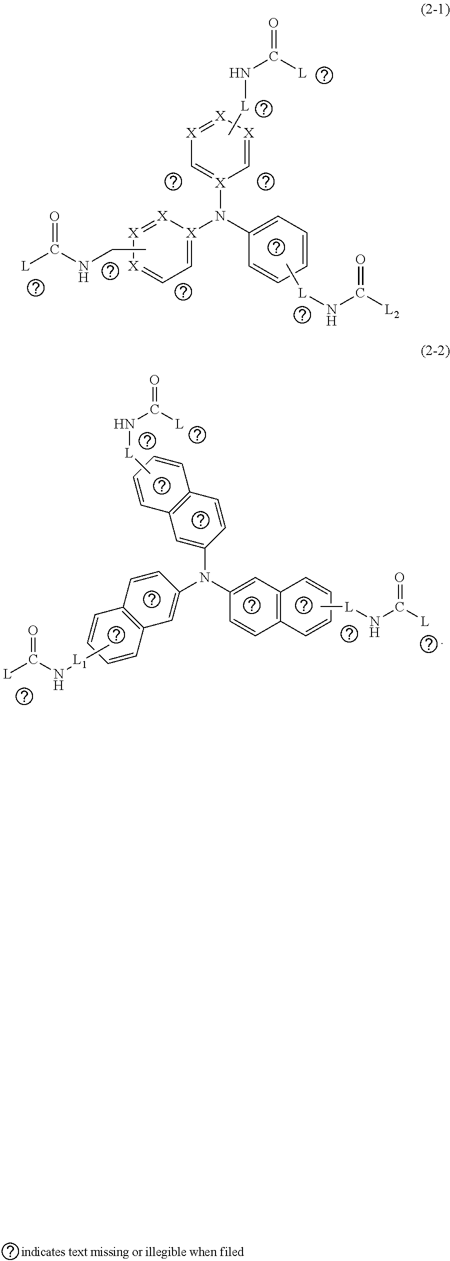

4. The organic compound according to claim 3, wherein each time Ar₁ appears is independently selected from an aryl group having 6 to 10 ring atoms that is unsubstituted or substituted with at least one R.

5. The organic compound according to claim 3, wherein each time L₁ appears is independently selected from a single bond, a C₁-C₈ linear hydrocarbon group unsubstituted or substituted with at least one R, a C₃-Cs branched hydrocarbon group unsubstituted or substituted with at least one R.

6. The organic compound according to claim 3, wherein each time L₂ appears is independently selected from C₁-C₃₀ halogenated hydrocarbon group.

7. The organic compound according to claim 1, wherein the organic compound is selected from any of the following structures:

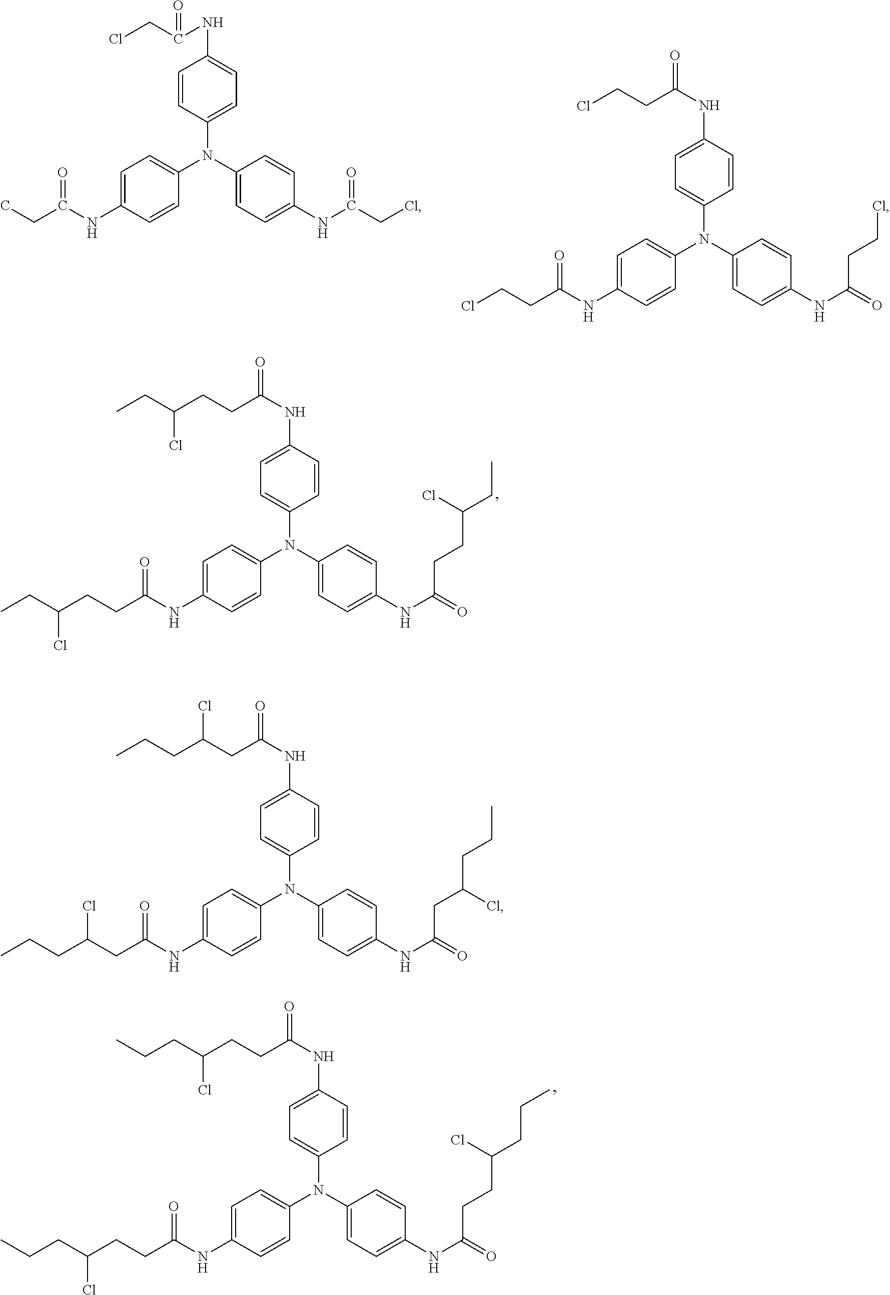 [SMILES: CCC(=O)Nc1ccc(N(c2ccc(NC(=O)CCl)cc2)c2ccc(NC(=O)CCl)cc2)cc1 ; O=C(CCCl)Nc1ccc(N(c2ccc(NC(=O)CCCl)cc2)c2ccc(NC(=O)CCCl)cc2)cc1 ; CCC(Cl)CCC(=O)Nc1ccc(N(c2ccc(NC(=O)CCC(Cl)CC)cc2)c2ccc(NC(=O)CCC(Cl)CC)cc2)cc1 ; CCCC(Cl)CC(=O)Nc1ccc(N(c2ccc(NC(=O)CC(Cl)CCC)cc2)c2ccc(NC(=O)CC(Cl)CCC)cc2)cc1 ; CCCC(Cl)CCC(=O)Nc1ccc(N(c2ccc(NC(=O)CCC(Cl)CCC)cc2)c2ccc(NC(=O)CCC(Cl)CCC)cc2)cc1]

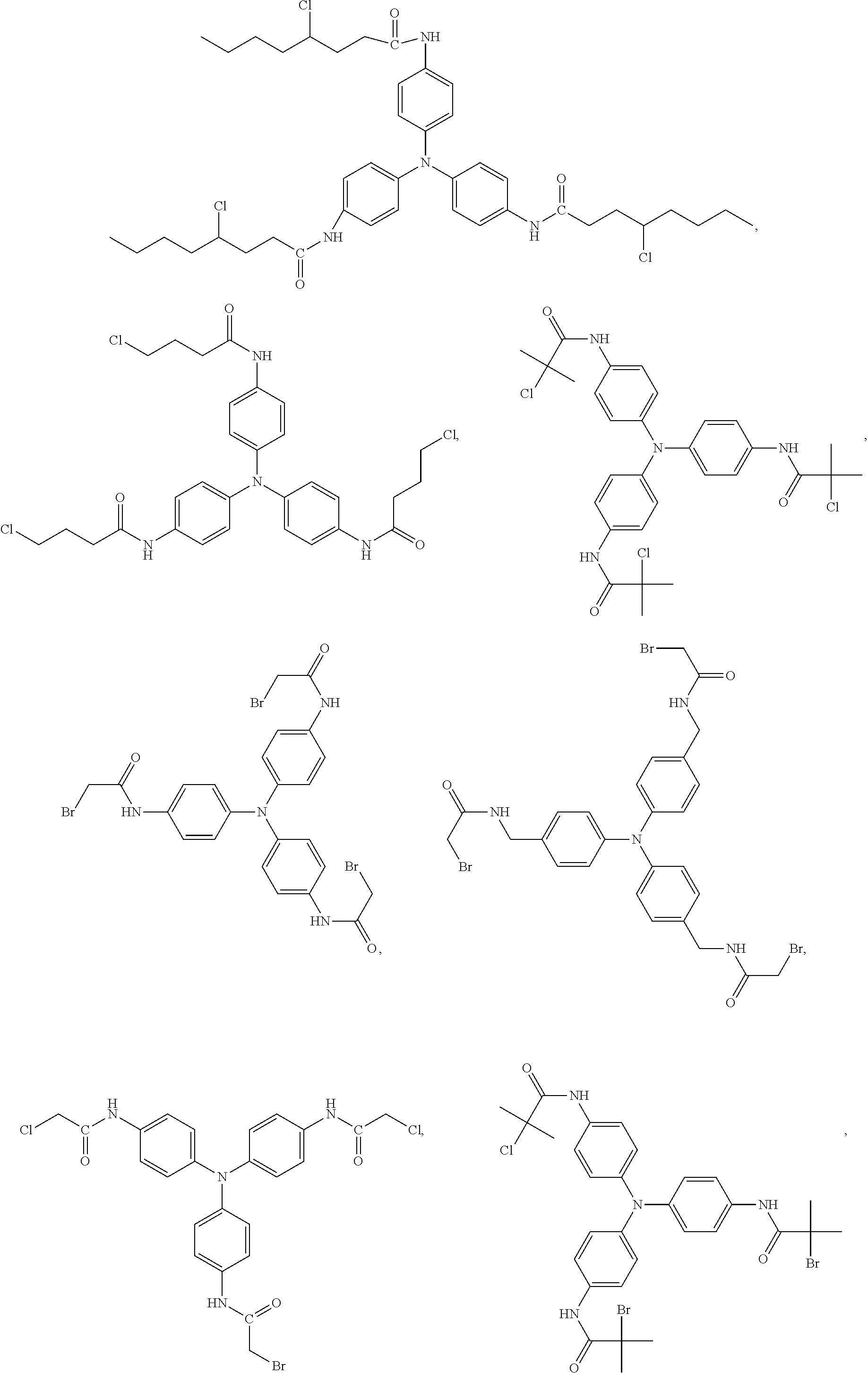

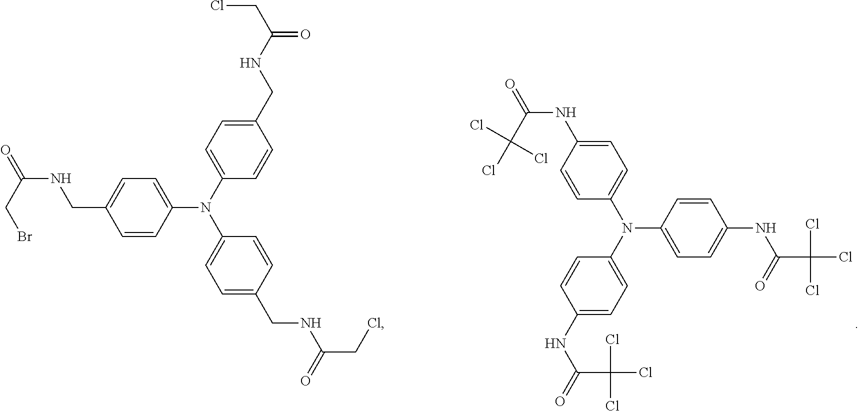 [SMILES: O=C(CCl)NCc1ccc(N(c2ccc(CNC(=O)CCl)cc2)c2ccc(CNC(=O)CBr)cc2)cc1 ; O=C(Nc1ccc(N(c2ccc(NC(=O)C(Cl)(Cl)Cl)cc2)c2ccc(NC(=O)C(Cl)(Cl)Cl)cc2)cc1)C(Cl)(Cl)Cl]

8. A method of synthesizing an organic compound, comprising:
mixing a first compound and a second compound in a first solvent to react to obtain an organic compound;
a structure of the first compound is represented by the general formula (I): 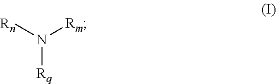
where each time Rₘ, Rₙ and R_{q} appear are each independently selected from 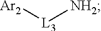
each time Ar₂ appears is independently selected from an aryl group having 6 to 30 ring atoms that is unsubstituted or substituted with at least one R, or a heteroaryl group having 5 to 30 ring atoms that is unsubstituted or substituted with at least one R, or any combination of these groups; a heteroatom of the heteroaryl group is selected from one or more of N, S, O, P, and Si, and a number of the heteroatom of the heteroaryl group is selected from 1 to 20;
each time L₃ appears is independently selected from a single bond, a C₁-C₃₀ aliphatic chain hydrocarbon group unsubstituted or substituted with at least one R, a hydroxyl group, an aldehyde group, a sulfhydryl group, an ether group, an ester group, an amino group, a carbonyl group, a nitro group, or a halogen;
each time R appears is independently selected from deuterium, a halogen, a hydroxyl group, a carboxyl group, a nitro group, a sulfonyl group, a sulfhydryl group, a cyano group, an aldehyde group, an amide group, or any combination of these groups;
a structure of the second compound is represented by the general formula (II): 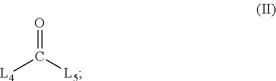
where L₄ is selected from a C₁-C₃₀ aliphatic chain hydrocarbon group unsubstituted or substituted with at least one R, a hydroxyl group, an aldehyde group, a sulfhydryl group, an ether group, an ester group, an amino group, a carbonyl group, or a nitro group;
L₅ is selected from a halogen;
each time R appears is independently selected from deuterium, a halogen, a hydroxyl group, a carboxyl group, a nitro group, a sulfonyl group, a sulfhydryl group, a cyano group, an aldehyde group, an amide group, or any combination of these groups.

9. The method according to claim 8, wherein each time Ar₂ appears is independently selected from the following groups: 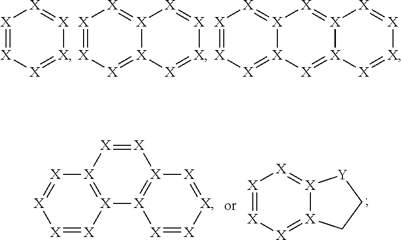
where each time X appears is independently selected from CR₁ or NR₁; each time R₁ appears is independently selected from hydrogen, deuterium, a halogen, a hydroxyl group, a carboxyl group, a nitro group, a sulfonyl group, a sulfhydryl group, a cyano group, an aldehyde group, an amide group, a C₁-C₃₀ aliphatic chain hydrocarbon group unsubstituted or substituted with at least one R, a C₁-C₃₀ aliphatic chain hydrocarbon oxy group unsubstituted or substituted with at least one R, a C₃-C₃₀ aliphatic cyclohydrocarbon group unsubstituted or substituted with at least one R, a C₃-C₃₀ aliphatic heterocyclohydrocarbon group unsubstituted or substituted with at least one R, an aryl group having 6 to 30 ring atoms that is unsubstituted or substituted with at least one R, an aryloxy group having 6 to 30 ring atoms that is unsubstituted or substituted with at least one R; a heteroaryl group having 5 to 30 ring atoms that is unsubstituted or substituted with at least one R, or heteroaryloxy group having 5 to 30 ring atoms that is unsubstituted or substituted with at least one R, or any combination of these groups; heteroatoms in the aliphatic heterocyclic hydrocarbyl group, the heteroaryl group, and the heteroaryloxy group are each independently selected from one or more of N, S, O, P, and Si, and numbers of heteroatoms in the aliphatic heterocyclic hydrocarbyl group, the heteroaryl group, and the heteroaryloxy group are each independently selected from 1 to 20;
each time Y appears is independently selected from S, N, O, a C₁-C₃₀ aliphatic chain hydrocarbon group unsubstituted or substituted with at least one R, a C₃-C₃₀ aliphatic cyclic hydrocarbon group unsubstituted or substituted with at least one R, or a C₃-C₃₀ aliphatic heterocyclic hydrocarbon group unsubstituted or substituted with at least one R.

10. The method according to claim 8, wherein a third compound is further provided, and the third compound is further provided, and the third compound is mixed with the first compound and the second compound in the first solvent, the third compound comprises an organic alkali.

11. The method according to claim 8, wherein the first solvent comprises an organic solvent.

12. The method according to claim 7, wherein the general formula (1) is selected from general formula (2-1) or general formula (2-2):

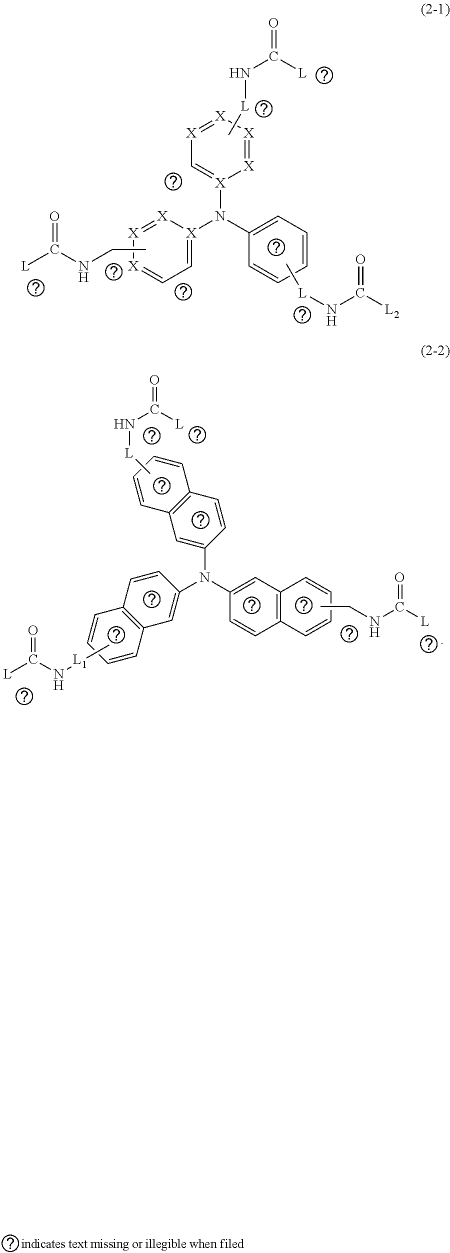

13. The method according to claim 12, wherein each time Ar₂ appears is independently selected from an aryl group having 6 to 10 ring atoms that is unsubstituted or substituted with at least one R;
each time L₃ appears is independently selected from a single bond, a C₁-C₈ linear hydrocarbon group unsubstituted or substituted with at least one R, or a C₃-C₈ branched hydrocarbon group unsubstituted or substituted with at least one R; and
each time L₄ appears is independently selected from a C₁-C₃₀ halogenated hydrocarbon group.

14. The method according to claim 10, wherein the third compound is selected from NR₁R₂R₃, where each time R₁, R₂, and R₃ appear are each independently selected from hydrogen, deuterium, or a C₁-C₃₀ aliphatic chain hydrocarbon group unsubstituted or substituted with at least one R.

15. The method according to claim 8, wherein before the step of mixing a first compound and a second compound in a first solvent, the method further comprises a step of synthesizing the first compound, and the step of synthesizing the first compound comprises:
providing a first precursor having a general formula (III) and a second precursor having a general formula (IV), dissolving the first precursor and the second precursor in a precursor solvent, and heating to obtain a first intermediate product;
dissolving a fourth compound and the first intermediate product in a second intermediate solvent to obtain the first compound, and the fourth compound is selected from one or more of hydrazine hydrate, borane, diisobutylaluminum hydride, lithium aluminum hydride, sodium borohydride, and potassium borohydride;
the general formula (III) is: 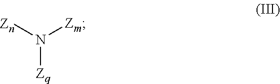
where each time Zₘ, Zₙ and Z_{q} appear are independently selected frome Ar₂-L₃;
the general formula (IV) is:

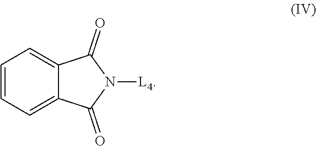

16. The method according to claim 15, wherein the step of dissolving the first precursor and the second precursor in a precursor solvent comprises: dissolving the first precursor, the second precursor, CuI, a strong alkali and weak acid salt, and 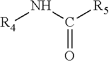
in a precursor solvent;
where each time R₄ and R₅ appears are each independently selected from a C₁-C₃₀ aliphatic chain hydrocarbon group unsubstituted or substituted with at least one R.

17. The method according to claim 16, wherein the precursor solvent is selected from one or more of 1,4-dioxane, dimethyl sulfoxide, toluene, and xylene.

18. The method according to claim 16, wherein the first solvent comprises dichloromethane, and a boiling point of the second intermediate solvent is less than or equal to 100° C.

19. An optoelectronic device, comprising:
a first electrode, a functional layer, and a second electrode stacked in this order;
wherein the functional layer comprises an organic compound comprising a compound represented by the general formula (1): 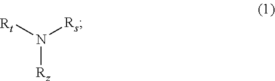
where, each time R_{z}, Rₛ and Rₜ appear are independently selected from 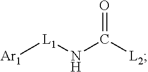
each time Ar₁ appears is independently selected from an aryl group having 6 to 30 ring atoms that is unsubstituted or substituted with at least one R, or a heteroaryl group having 5 to 30 ring atoms that is unsubstituted or substituted with at least one R, or any combination of these groups; a heteroatom of the heteroaryl group is selected from one or more of N, S, O, P, and Si, and a number of the heteroatom of the heteroaryl group is selected from 1 to 20;
each time L₁ appears is independently selected from a single bond, a C₁-C₃₀ aliphatic chain hydrocarbon group unsubstituted or substituted with at least one R, a hydroxyl group, an aldehyde group, a sulfhydryl group, an ether group, an ester group, an amino group, a carbonyl group, a nitro group, or a halogen;
each time L₂ appears is independently selected from a C₁-C₃₀ aliphatic chain hydrocarbon group unsubstituted or substituted with at least one R, hydroxyl group, an aldehyde group, a sulfhydryl group, an ether group, an ester group, an amino group, a carbonyl group, or a nitro group; and
each time R appears is independently selected from deuterium, a halogen, a hydroxyl group, a carboxyl group, a nitro group, a sulfonyl group, a sulfhydryl group, a cyano group, an aldehyde group, an amide group, or any combination of these groups.

20. The optoelectronic device according to claim 19, wherein the first electrode and the second electrode independently comprise, for example, but are not limited to, a doped metal oxide electrode, a composite electrode, a graphene electrode, a carbon nanotube electrode, a metal elemental electrode, or an alloy electrode; a material of the doped metal oxide electrode comprises one or more of indium-doped tin oxide, fluorine-doped tin oxide, antimony-doped tin oxide, aluminum-doped zinc oxide, gallium-doped zinc oxide, indium-doped zinc oxide, magnesium-doped zinc oxide, aluminum-doped magnesium oxide, and cadmium-doped zinc oxide; the composite electrode comprises AZO/Ag/AZO, AZO/Al/AZO, ITO/Ag/ITO, ITO/Al/ITO, ZnO/Ag/ZnO, ZnO/Al/ZnO, TiO₂/Ag/TiO₂, TiO₂/Al/TiO₂, ZnS/Ag/ZnS, ZnS/Al/ZnS, Ca/Al, LiF/Ca, LiF/Al, BaF₂/Al, CsF/Al, CaCO/Al, or BaF₂/Al; a material of the metal elemental electrode comprises one or more of Ag, Ni, Pt, Au, Ir, Cu, Mo, Al, Ca, Mg, and Ba; the alloy electrode comprises Au:Mg alloy electrode or Ag:Mg alloy electrode;
the functional layer comprises a hole functional layer, a modification layer, an excitation layer and an electronic functional layer stacked in this order;
a material of the hole functional layer comprises one or more of 4,4′-N,N′-dicarbazolyl-biphenyl, poly [bis(4-phenyl) (2,4,6-trimethylphenyl) amine], N,N′-diphenyl-N,N′-bis(1-naphthyl)-1,1′-biphenyl-4,4′-diamine, N,N′-diphenyl-N,N′-bis(3-methylphenyl)-(1,1′-biphenyl)-4,4′-diamine, poly (N,N′bis(4-butylphenyl)-N,N′-bis(phenyl) benzidine), N,N′-bis(3-methylphenyl)-N,N′-bis(phenyl)-spiro, N,N′-bis(4-(N,N′-diphenyl-amino) phenyl)-N,N′-diphenylbenzidine, 4,4′,4′-tris(N-carbazolyl)-triphenylamine, 4,4′,4′-tris(N-3-methylphenyl-N-phenylamino) triphenylamine, poly [(9,9′-dioctylfluorene-2,7-diyl)-co-(4,4′-(N-(4-sec-butylphenyl) diphenylamine)], poly (N-vinylcarbazole) and derivatives thereof, N,N′-bis(1-naphthyl)-N,N′-diphenyl-1,1′-biphenyl-4-4′-diamine, spiro NPB, poly (phenylenevinylene), poly [2-methoxy-5-(2-ethylhexyloxy)-1,4-phenylenevinylene], poly [2-methoxy-5-(3′,7′-dimethyloctoxy)-1,4-phenylenevinylene], 2,2′,7,7′-tetrakis [N,N-bis(4-methoxyphenyl) amino]-9,9′-spirobifluorene, 4,4′-cyclohexyl bis[N,N-bis (4-methylphenyl) aniline], 1,3-bis(carbazol-9-yl) benzene, polyaniline, polypyrrole, poly (p) phenylenevinylidene, aromatic tertiary amine, polynuclear aromatic tertiary amine, 4,4′-bis(p-carbazolyl)-1,1′-biphenyl compound, N,N,N′,N′-tetraarylbenzidine, PEDOT:PSS and derivatives thereof, polymethacrylates and derivatives thereof, poly (9,9-octylfluorene) and derivatives thereof, poly (spirofluorene) and derivatives thereof, 2,3,6,7,10,11-hexacyano-1,4,5,8,9,12-hexaazabenzophenanthrene, PEDOT, PEDOT:PSS doped with s-MoO₃ derivatives, 4,4′,4′-tris(N-3-methylphenyl-N-phenylamino) triphenylamine, tetracyanoquinone dimethane, doped graphene, undoped graphene, C60, copper phthalocyanine, a second doped metal oxide particle, a second undoped metal oxide particle, a metal sulphide, and a metal nitride;
the modification layer comprises the organic compound;
a material of the excitation layer comprises one or more of a single structure quantum dot, a core-shell structure quantum dot, and a perovskite-type semiconductor material;
a material of the single structure quantum dot, a material of the core of the core-shell structure quantum dot, and a material of the shell of the core-shell structure quantum dot comprise one or more of a Group II-VI compound, a Group IV-VI compound, a Group III-V compound, and a Group I-III-VI compound, respectively; the Group II-VI compound comprises one or more of CdS, CdSe, CdTe, ZnS, ZnSe, ZnTe, ZnO, HgS, HgSe, HgTe, CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, CdHgTe, HgZnS, HgZnSe, HgZnTe, CdZnSeS, CdZnSeTe, CdZnSTe, CdHgSeTe, CdHgSTe, HgZnSeS, HgZnSeTe, and HgZnSTe; the Group IV-VI compound comprises one or more of SnS, SnSe, SnTe, PbS, PbSe, PbTe, SnSeS, SnSeTe, SnSTe, PbSeS, PbSeTe, PbSTe, SnPbS, SnPbSe, SnPbTe, SnPbSSe, SnPbSeTe, and SnPbSTe; the Group III-V compound comprises one or more of GaN, GaP, GaAs, GaSb, AlN, AlP, AlAs, AlSb, InN, InP, InAs, InSb, GaNP, GaNAs, GaNSb, GaPAs, GaPSb, AlNP, AlNAs, AlNSb, AlPAs, AlPSb, InNP, InNAs, InNSb, InPAs, InPSb, GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, and InAlPSb; the Group I-III-VI compound comprises one or more of CuInS₂, CuInSe₂, and AgInS₂;
the perovskite-type semiconductor material comprises a doped or undoped inorganic perovskite-type semiconductors, or an organic-inorganic hybrid perovskite-type semiconductor; a general structural formula of the inorganic perovskite-type semiconductor is AMX₃, where A is a Cs⁺ ion, M is a divalent metal cation comprising one or more of Pb²⁺, Sn²⁺, Cu²⁺, Ni²⁺, Cd²⁺, Cr²⁺, Mn²⁺, Co²⁺, Fe²⁺, Ge²⁺, Yb²⁺, and Eu²⁺, and X is a halogen anion comprising one or more of Cl⁻, Br⁻, and I⁻; a general structural formula of the organic-inorganic hybrid perovskite-type semiconductor is BMX₃, where B is an organic amine cation comprising CH₃(CH₂)ₙ₋₂NH₃⁺ or [NH₃(CH₂)ₙNH₃]²⁺, where n≥2, M is a divalent metal cation comprising one or more of Pb²⁺, Sn²⁺, Cu²⁺, Ni²⁺, Cd²⁺, Cr²⁺, Mn²⁺, Co²⁺, Fe²⁺, Ge²⁺, Yb²⁺, and Eu²⁺, and X is a halogen anion comprising one or more of Cl⁻, Br⁻, and I⁻; and
a material of the electronic functional layer comprises one or more of an inorganic electronic functional material and an organic electronic functional material, the inorganic electronic functional material comprises one or more of a first doped metal oxide particle, a first undoped metal oxide particle, a Group IIB-VIA semiconductor material, a Group IIIA-VA semiconductor material, and a Group IB-IIIA-VIA semiconductor material; the organic electronic functional material comprises one or more of a quinoxaline compound, an imidazole compound, a triazine compound, a fluorene-containing compound, a hydroxyquinoline compound, and a fullerene derivative.
